(19) United States
(12) Patent Application Publication
(10) Pub. No.: US 2026/0347936 A1
(43) Pub. Date: Nov. 5, 2026

(54) METHODS FOR DETERMINING SEPSIS SEVERITY AND TREATING SEPSIS USING DRP1-RELATED GENES

(71) Applicants: Ting WANG, MIAMI, FL (US); Stephen BLACK, MIAMI, FL (US); Anlin FENG, MIAMI, FL (US); Marissa POKHAREL, MIAMI, FL (US)

(72) Inventors: Ting WANG, MIAMI, FL (US); Stephen BLACK, MIAMI, FL (US); Anlin FENG, MIAMI, FL (US); Marissa POKHAREL, MIAMI, FL (US)

(73) Assignee: THE FLORIDA INTERNATIONAL UNIVERSITY BOARD OF TRUSTEES, MIAMI, FL (US)

(21) Appl. No.: 19/194,807

(22) Filed: Apr. 30, 2025

(51) Int. Cl. C12Q 1/6883

(52) U.S. Cl. CPC C12Q 1/6883; C12Q 2600/118; C12Q 2600/158

(57) ABSTRACT

The subject invention provides a Drp1-associated gene signature for sepsis prognosis, and also a method of characterizing sepsis survival risk in a subject by a sepsis survival score calculated as a linear combination of gene expression values and their corresponding weight values in the Drp1-associated gene signature. Based on the sepsis survival score calculated for a subject, the subject invention further provides methods of determining sepsis severity, determining efficacy of a therapy for sepsis, and treating sepsis in a subject.

### GOVERNMENT SUPPORT

This invention was made with government support under P01 HL134610 awarded by the National Institutes of Health. The government has certain rights in the invention.

### BACKGROUND

Sepsis is a deadly syndrome involving many dysregulated pathways in response to infection or injury, ultimately leading to organ dysfunction and damage. Sepsis remains a major public health concern due to its high incidence, mortality rate, and strain on the hospital care system. The mortality rate is estimated to be 41.9% for intensive care unit (ICU) septic patients. Unfortunately, increasing sepsis survival is not the only hurdle in treating sepsis; but quality of life after sepsis is a major concern. It is estimated that nearly 75% of sepsis survivors have deficits in at least one activity of daily life.

Currently, the only treatment for sepsis is the administration of intravenous broad-spectrum antibiotics, with the administration preferably within an hour of sepsis recognition. Sepsis survival drops 7.6% every hour that administration of antibiotics is delayed. This short window for antibiotic administration means early detection of sepsis is critical.

Sepsis treatment also includes supportive care, which involves the initiation of vasopressors and aggressive fluid therapy. However, supportive care only offers limited/modest success. This means that the only proven treatment to reduce sepsis mortalities is early detection and immediate administration of antibiotics. Unfortunately, there are no reliable biomarkers or metrics that have been proven to detect sepsis early. Therefore, the only recourse for clinicians is to perform repeated blood cultures to monitor serum lactic acid levels and blood pressure in the hope of identifying sepsis or septic shock cases early, causing significant strain on hospital resources. Septic shock is the most severe form of sepsis and patients with septic shock have dire metabolic and cellular abnormalities that are associated with higher mortality rates than sepsis alone.

Historically, sepsis progression has been divided into two inflammatory stages. Originally, the first stage was believed to be a pro-inflammatory stage where a cytokine storm induces a profound and intense immune response, followed by a compensatory, anti-inflammatory stage. Early investigations targeted the presumed first, pro-inflammatory stage and evaluated anti-inflammatory treatments. These strategies were unsuccessful in clinical trials. Newer evidence suggests that sepsis is not biphasic, with a distinct pro-inflammatory stage followed by an anti-inflammatory stage, but rather occurs simultaneously. This is likely due to the complexity of immune responses, where pathways constantly interact and modulate one another.

Immunomodulation has been the predominant target tested in sepsis clinical trials. Despite over 100 randomized clinical trials testing immunomodulation, they have failed to yield even one successful drug or strategy. This failure likely stems from the cross-talk of the immune response; thus, simply augmenting or inhibiting specific immunomodulators does not have the intended effect as the system responds to this modification. Although further testing is needed to verify why immunomodulation has failed to clinically demonstrate efficacy, the overwhelming failure of immunomodulatory clinical trials emphasizes that this is not a valid strategy for sepsis treatment; therefore, research effort needs to be allocated to other approaches.

Sepsis is known to be the cumulation of multiple dysregulated pathways; therefore, it is critical to identify therapeutics that target multiple pathways. Mitochondria are known to play critical roles in a multitude of pathways involved in maintaining homeostasis. For example, mitochondria supply the energy and metabolic intermediates that are needed for immune cell activation and function and influence inflammatory and cell death pathways. Dysfunctional mitochondria are well documented in sepsis, although modulation of mitochondria remains primarily untested.

Mitochondria are highly dynamic organelles that exist in networks rather than individually. Mitochondrial networks can modulate their function by combining (fusion) or separating (fission). The cell contains a multitude of mitochondrial networks that are constantly performing fusion or fission to modulate their function to adapt to cellular demands, a process referred to as mitochondrial dynamics.

Despite immense research efforts, there are still no available therapeutics for treating sepsis other than general antibiotics and source control. Early recognition and action are the only courses of action for physicians to improve patient outcomes. This necessitates a quick diagnosis to reduce sepsis mortality. Unfortunately, the identification of patients at risk of septic death may not be sufficient to reduce mortality rates. This is exemplified in a retrospective cohort study of 6 hospitals between 2014 and 2015, where only 3.7% of sepsis-associated deaths were considered to be either moderately or definitively preventable. This suggests that even early identification of high-risk patients and subsequent implementation of aggressive treatments will only minimally improve sepsis outcomes. This highlights the urgency to identify new strategies to test and implement to improve sepsis survival.

Despite advances in medical treatment, current therapies are often inadequate, with high septic mortality rates. Therefore, there is a critical need for reliable prognostic markers and new therapeutic strategies to be used in clinical settings to improve the management and outcomes of patients with sepsis.

### BRIEF SUMMARY

The present invention provides new materials and methods for sepsis diagnosis, prognosis, and/or treatment, and further develops a system for determining a survival risk for each subject based on, for example, measurements of the biomarkers. Advantageously, this system allows for the classification of subjects into high or low sepsis survival score cohorts, and can accurately assign treatment outcomes, enabling personalized therapeutic strategies tailored to individual risk profiles.

In one embodiment, biomarkers provided according to the present invention can be detected in easily obtainable samples from the subject, such as blood. By characterizing sepsis survival risk using the system, further applications, such as adopting personalized therapy strategies for sepsis, evaluating the effectiveness of new therapeutic devices, strategies, or treatments for sepsis, are also enabled by the present invention, which improves sepsis survival.

In some embodiments, the present invention provides a dynamin-related protein 1 (Drp1, gene code DNM1L)-associated gene signature for sepsis prognosis, the Drp1-associated gene signature comprises at least one of, for example, AFG3L2, BCL2, ITPR3, LONP1, OPTN, PHB, and TOMM40, and/or at least one of, for example, SLC4A7, PDCD2L, UPP1, ARL4C, NELL2, LEF1, SGK1, PDE4D, S1PR3, MCTP2, HES6, and OLR1.

In some embodiments, the present invention provides a method of characterizing sepsis survival risk in a subject by a sepsis survival score, which is calculated as a linear combination of gene expression values and their corresponding weight values in the Drp1-associated gene signature described herein. In a preferred embodiment, the sepsis survival score is calculated using a formula of:

$$sepsis survival score = {\sum }_{i = 1}^{n} {W}_{i} ( \frac{{e}_{i} - {μ}_{i}}{{S}_{i}} ) ,$$

wherein n is the number of genes in the gene signature; Wᵢ is the weighted value of each gene; eᵢ is the expression level of each gene; and μᵢ and Sᵢ are the mean and standard deviation of the gene expression level values, respectively, for the corresponding gene compared the whole sample.

The sepsis survival score is calculated based on at least one gene selected from the Drp1-associated gene signature described herein, and is indicative of the probability of survival from sepsis for a subject, or the probability of septic death for a subject. In some embodiments, a subject with a sepsis survival score that is higher than the mean/medium sepsis survival score is classified as having low risk of septic death, while a subject with a sepsis survival score that is lower than the mean/medium sepsis survival score is classified as having high risk of septic death. In a specific embodiment, the mean/medium sepsis survival score is 0.

In some embodiments, the present invention provides a method of determining sepsis severity in a subject, the method comprising the following steps:
(1) obtaining a sample from the subject, which preferably comprises obtaining peripheral blood from the subject, and optionally separating PBMCs therefrom;
(2) detecting in the sample the expression level of at least one gene selected from a Drp1-associated gene signature, wherein the Drp1-associated gene signature comprises at least one of AFG3L2, BCL2, ITPR3, LONP1, OPTN, PHB, and TOMM40, and/or at least one of SLC4A7, PDCD2L, UPP1, ARL4C, NELL2, LEF1, SGK1, PDE4D, S1PR3, MCTP2, HES6, and OLR1;
(3) calculating the sepsis survival score as a linear combination of gene expression values and their corresponding weight values in the Drp1-associated gene signature; and
(4) determining sepsis severity in the subject according to the sepsis survival score, comprising comparing the sepsis survival score calculated by step (3) with a mean/medium sepsis survival score, and determining the subject as having a high sepsis severity if the sepsis survival score as calculated is lower than the mean/medium sepsis survival score, while determining the subject as having a low sepsis severity if the sepsis survival score as calculated is higher than the mean/medium sepsis survival score. In a specific embodiment, the mean/medium sepsis survival score is 0.

In some embodiments, the present invention provides a method of determining efficacy of a therapy for sepsis in a subject in need thereof, wherein the subject is one that has been diagnosed with sepsis, comprising the following steps:
(1) obtaining a sample from the subject, detecting in the sample the expression level of at least one gene selected from a Drp1-associated gene signature, and calculating a sepsis survival score as a linear combination of gene expression values and their corresponding weight values in the Drp1-associated gene signature, wherein the Drp1-associated gene signature comprises at least one of AFG3L2, BCL2, ITPR3, LONP1, OPTN, PHB, and TOMM40, and/or at least one of SLC4A7, PDCD2L, UPP1, ARL4C, NELL2, LEF1, SGK1, PDE4D, S1PR3, MCTP2, HES6, and OLR1;
(2) administering the therapy to the subject;
(3) obtaining a further sample from the subject, detecting in the further sample the expression level of at least one gene selected from a Drp1-associated gene signature, and calculating a sepsis survival score as a linear combination of gene expression values and their corresponding weight values in the Drp1-associated gene signature, wherein the samples obtained in step (1) and step (3) are of the same type, and/or wherein the Drp1-associated gene signature comprises at least one of AFG3L2, BCL2, ITPR3, LONP1, OPTN, PHB, and TOMM40, and/or at least one of SLC4A7, PDCD2L, UPP1, ARL4C, NELL2, LEF1, SGK1, PDE4D, S1PR3, MCTP2, HES6, and OLR1, and/or wherein the genes whose expression levels are detected in step (1) and step (3) are the same group of genes; and
(4) determining efficacy of the therapy for sepsis in the subject, comprising comparing the sepsis survival score calculated in step (3) with the sepsis survival score calculated in step (1), and determining the therapy as being effective for sepsis in the subject if the sepsis survival score calculated in step (3) is higher than the sepsis survival score calculated in step (1), while determining the therapy as being ineffective for sepsis in the subject if the sepsis survival score calculated in step (3) is equal to or lower than the sepsis survival score calculated in step (1).

In some embodiments, the present invention provides a method of treating sepsis in a subject in need thereof, wherein the subject is one that has been diagnosed with sepsis, the method comprising the following steps:
(1) administering a therapy for sepsis to the subject;
(2) at any point of sepsis treatment, obtaining a sample from the subject, detecting in the sample the expression level of at least one gene selected from a Drp1-associated gene signature, and calculating a sepsis survival score as a linear combination of gene expression values and their corresponding weight values in the Drp1-associated gene signature, wherein step (2) is performed repeatedly at least twice throughout the sepsis treatment, and/or wherein the Drp1-associated gene signature comprises at least one of AFG3L2, BCL2, ITPR3, LONP1, OPTN, PHB, and TOMM40, and/or at least one of SLC4A7, PDCD2L, UPP1, ARL4C, NELL2, LEF1, SGK1, PDE4D, S1PR3, MCTP2, HES6, and OLR1, and/or wherein the genes whose expression levels are detected in step (2) are the same group of genes for each repetition of step (2) in the subject; and
(3) modulating treatment strategy according to alteration of the sepsis survival score, comprising strengthening or changing treatment strategy, such as increasing treatment dosage and/or frequency, or changing to a new treatment, if the sepsis survival score decreases or keeps unchanged among at least two repetitions of step (2); and maintaining or attenuating treatment strategy, such as decreasing treatment dosage and/or frequency, if the sepsis survival score increases among at least two repetitions of step (2).

In one embodiment, the present invention provides a method of treating sepsis in a subject in need thereof, comprising the following steps:
(1) obtaining a sample from the subject;
(2) detecting in the sample the expression level of at least one gene selected from AFG3L2, BCL2, ITPR3, LONP1, OPTN, PHB, and TOMM40, and/or SLC4A7, PDCD2L, UPP1, ARL4C, NELL2, LEF1, SGK1, PDE4D, S1PR3, MCTP2, HES6, and OLR1;
(3) calculating a first sepsis survival score as a linear combination of gene expression values and their corresponding weight values of said gene(s);
(4) treating the subject for sepsis;
(5) obtaining a sample from the subject after treatment for sepsis;
(6) detecting in the sample the expression level of at least one gene selected from AFG3L2, BCL2, ITPR3, LONP1, OPTN, PHB, and TOMM40, and/or SLC4A7, PDCD2L, UPP1, ARL4C, NELL2, LEF1, SGK1, PDE4D, S1PR3, MCTP2, HES6, and OLR1;
(7) calculating a second sepsis survival score as a linear combination of gene expression values and their corresponding weight values of said gene(s) after treatment for sepsis; and
(8) determining the treatment as a favorable or effective treatment if the sepsis survival score increases after the treatment, or determining the treatment as an unfavorable or ineffective treatment if the sepsis survival score remains unchanged or decreases after the treatment; or
determining the treatment as a favorable or effective treatment if the second sepsis survival score is higher than the first sepsis survival score, or determining the treatment as an unfavorable or ineffective treatment if the second sepsis survival score is equal to or lower than the first sepsis survival score.

In one embodiment, the subject invention provides a method of reducing sepsis severity in a subject, the method comprising the following steps:
obtaining a sample from the subject;
detecting in the sample the expression level of at least one gene selected from at least one Drp1-associated gene signature;
calculating a first sepsis survival score as a linear combination of gene expression values and their corresponding weight values in the at least one Drp1-associated gene signature;
treating the subject for sepsis;
obtaining a sample from the subject after the treatment;
detecting in the sample after the treatment the expression level of at least one gene selected from at least one Drp1-associated gene signature;
calculating a second sepsis survival score as a linear combination of gene expression values and their corresponding weight values in the at least one Drp1-associated gene signature;
determining a reduced sepsis severity in the subject if the second sepsis survival score is higher than the first sepsis survival score.

### BRIEF DESCRIPTION OF DRAWINGS

FIG. 1 shows an overview of gene signature (GS) generation. First, Drp1-associated genes were compared to sepsis survival genes and 12 common genes were identified, establishing a GS termed as DEG-based GS. Next, genes of proteins that interact with Drp1 were compared to sepsis survival genes and 7 common genes were identified, establishing a GS termed as PPI-based GS.

FIGS. 2A-2H show differentially expressed genes and protein-protein interaction analysis reveal a 12 and 7 Drp1-associated gene signature that corresponds with sepsis survival genes. 12 of 438 Drp1-associated genes (generated by identifying DEG after Drp1 overexpression) match genes related to sepsis survival (2A), these 12 genes are referred to as DEG-based GS. PPI analysis of Drp1 identified 244 Drp1-associated genes. 7 of these genes are common between the Drp1-associated genes and sepsis survival genes (2B); these 7 genes are referred to as PPI-based GS. KEGG analysis was used to identify pathways enriched with both Drp1-associated GS (2C) and pathways enriched with the sepsis survival genes (2D). The correlation matrix of Drp1-DGEs (2E) and Drp1-PPI GS (2F) shows the relationship of individual genes compared to the other genes in the GS. PPI network for the genes in the PPI-based GS (2G), and DEG-based GS have no interactions. Genomic locations for both GS are shown in a circular plot (2H), with the DEG-based GS represented by blue lettering, and the PPI-based GS represented by red lettering. Red dots represent upregulated genes and blue dots represent downregulated genes related to sepsis survival.

FIGS. 3A-3F show Drp1-associated gene signatures differentiate between low and high-risk sepsis groups in both the discovery and validation cohort. Heatmap of the DEG-based (3A) and PPI-based GS (3B) reveal expression patterns that differentiate low and high-risk sepsis groups. Violin plots of the sepsis survival scores from DEG-based (3C) and the PPI-based (3D) GS show that these gene signatures successfully differentiate between low and high-risk sepsis patients in both the discovery and validation cohorts. ROC curves of the DEG-based (3E) and PPI-based (3F) GS exhibit the sensitivity and specificity of these gene signatures.

FIGS. 4A-4D show PCA and density distribution plots of both Drp1-associated gene signatures. PCA of both DEG-based (4A) and PPI-based (4C) GS were performed to reduce dimensionality and assess the similarity between each individual sample. The PCA shows that both gene signatures can entirely differentiate high-risk sepsis patients and low-risk sepsis patients. The PCA analysis for DEG-based GS represents 78.5-84.2% of the variable expression data. The PCA analysis for PPI-based GS represents 52.6-59.3% of the variable expression data. The density distribution of the AUC of random genes (blue) and sepsis survival-related genes (yellow) show that both DEG-based GS (4B) and PPI-based GS (4D) have a prognostic power greater than random genes from the whole genome. The PPI-based GS (4D) prognostic power is greater than that of other random genes related to sepsis survival. The sum of the DEG-based GS AUC value (1.58) and the sum of the PPI-based GS AUC value (1.88) is indicated by the red inverted triangles.

FIGS. 5A-5F show analysis of low- and high-scores obtained from PPI-based GS. KEGG analysis of the upregulated and downregulated DEGs from high-score, lower-risk patients in the discovery cohort (5A), using the Drp1-PPI GS to generate the sepsis survival scores (Tables 4-5). Gene set variation analysis (GSVA) shows the log FC of the pathways enriched in the DEGs from the high-score, lower-risk cohort (5B). The heatmap generated from the GSVA shows pathway expression patterns that differentiate the low- and high-score cohorts (5C). GSEA shows that the high-score, lower-risk cohort has increased T cell receptor signaling and decreased expression of metabolic pathways (5D). Using CIBERSORT, immune cell proportions in PBMCs were estimated in the entire discovery cohort (5E). Separating the low- and high-risk cohorts shows a predicted immune profile shift between the groups (5F). Specifically, the high-score, lower-risk cohort has decreased levels of neutrophils and increased activation of NK cells.

FIG. 6 shows illustrations of clinical use of the present invention.

### DETAILED DESCRIPTION

The present invention provides new materials and methods for sepsis diagnosis, prognosis, and/or treatment. The present invention also provides a system for determining a survival risk for each subject based on, for example, measurements of the biomarkers. Advantageously, this system allows for the classification of subjects into high or low sepsis survival score cohorts, and can accurately assign treatment outcomes, enabling personalized therapeutic strategies tailored to individual risk profiles.

Specifically, in accordance with the present invention, a 12-gene signature (GS) was established as a differentially expressed gene (DEG)-based GS. Next, genes of proteins that interact with Drp1 were compared to sepsis survival genes and 7 common genes were identified, establishing a GS termed as protein-protein interaction (PPI)-based GS in the present invention. To evaluate if these GSs can predict sepsis survival, publicly available human blood transcriptomic datasets from sepsis patients were used. Both GSs can successfully predict sepsis survival in both discovery and validation cohorts with high sensitivity and specificity, with the PPI-based GS showing enhanced prognostic performance.

The present invention provides new and validated blood-borne biomarkers (PPI-based 7-gene GS) for sepsis survival risk prediction. These biomarkers can be used for improving the early identification of high-risk sepsis patients and optimizing personalized treatment strategies to reduce sepsis mortality.

In one embodiment, the subject invention also provides compositions for use in the methods of the subject invention. In certain embodiments, the composition comprises one or more of the following:
probes, and/or amplification oligonucleotides for, for example, detecting one or more genes in the Drp1-associated gene signatures;
antibodies, and/or reporter labels for, for example, detecting one or more proteins selected from Drp1 and proteins expressed from genes in the Drp1-associated gene signatures; and/or
reagents, buffers, and/or controls for performing the methods of the subject invention.

In one embodiment, the subject invention further provides a kit comprising all necessary components for perform the methods of the subject invention, including, for example, the composition of the subject invention, and instructions. The kits described herein find use in research, therapeutic, screening, and clinical applications.

Reference now will be made to certain detailed aspects of various embodiments of the invention. It is to be understood that the disclosed embodiments are merely exemplary of the invention that may be embodied in numerous and alternative forms. Therefore, specific details disclosed herein are not to be interpreted as limiting, but merely as a representative basis for any aspect of the invention and/or as a representative basis for teaching one skilled in the art to variously employ the invention.

### DEFINITIONS

To facilitate an understanding of the present invention, a number of terms and phrases are defined below. The terminology used herein is used only for the purpose of describing particular embodiments of the present invention and is not intended to be limiting in any way.

As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, and “comprise” can be used interchangeably; “consisting essentially of”, and “consists essentially of” can be used interchangeably; and “consisting”, and “consists” can be used interchangeably.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

Except in the examples, or where otherwise expressly indicated, all numerical quantities in this description indicating amounts of material and/or use are to be understood as modified by the word “about” in describing the broadest scope of the invention. Practice within the numerical limits stated is generally preferred.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed. In the context of compositions containing amounts of concentrations of ingredients where the term “about” is used, these values include a variation (error range) of 0-10% around the value (X±10%).

Ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

Throughout this application, where publications are referenced, the disclosures of these publications in their entireties are hereby referred to in this application to more fully describe the state of the art to which this invention pertains.

The term “sepsis survival” means survival from sepsis and/or sepsis-associated symptoms including but being not limited to inflammation. The term “septic death” means death from sepsis and/or sepsis-associated symptoms including but being not limited to inflammation.

The terms “detect”, “detecting” or “detection” may describe either the general act of discovering or discerning or the specific observation of a detectably labeled composition.

The term “subject” refers to any organisms that are screened using the diagnostic methods described herein. Such organisms refer to an animal, preferably include, but are not limited to, mammals (e.g., humans). Non-limiting examples of subjects include canine, porcine, rodent, feline, bovine, poultry, equine, human, and a non-human primate. The subject can be of any age or stage of development, including infant, toddler, adolescent, teenager, adult, or senior.

The term “diagnosed”, “diagnosis” or “diagnostic” refers to the recognition of a disease by its signs and symptoms, or genetic analysis, pathological analysis, histological analysis, and the like.

The term “nucleic acid” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragments are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences”. Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

The term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized”.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

The term “sample” is used in its broadest sense. In one sense, it is meant to include a specimen or culture obtained from any source, as well as biological and environmental samples. Biological samples may be obtained from animals (including humans) and encompass fluids, solids, tissues (e.g., biopsy samples), cells, and gases. Biological samples include blood products, such as plasma, serum and the like. Such examples are not, however, to be construed as limiting the sample types applicable to the present invention.

The terms “treatment” or any grammatical variation thereof (e.g., treat, treating, etc.), as used herein, includes but is not limited to, the application or administration to a subject (or application or administration to a cell or tissue from a subject) with the purpose of slowing, stabilizing, curing, healing, alleviating, relieving, remedying, less worsening, ameliorating, or improving the disease or condition. The term “treating” refers to any indication of success in the treatment or amelioration of a pathology or condition, including any objective or subjective parameter such as abatement; remission; lessening of the rate of worsening; lessening severity of the disease; stabilization, diminishing of symptoms or making the pathology or condition more tolerable to the subject; or improving a subject's physical or mental well-being.

Abbreviations used herein include: Drp1: dynamin-related protein 1; DEGs: differentially expressed genes; PPI: protein-protein interaction; GS: gene signature; KEGG: Kyoto Encyclopedia of Genes and Genomes; FDR: false discovery rate, or false discovery rate adjusted p-value; IR: immune response; ROC: receiver operating characteristic curve; PCA: principal component analysis; AUC: area under the curve; FC: fold change; GSVA: gene set variation analysis; GSEA: gene set enrichment analysis; PBMC: peripheral blood mononuclear cell.

### Biomarkers of Sepsis

Novel biomarkers are provided by the present invention for sepsis diagnosis, sepsis prognosis, and/or sepsis treatment, particularly for predicting sepsis severity, sepsis survival, and/or for designing treatment.

In some embodiments, the present invention provides protein biomarkers for sepsis prognosis. The protein biomarkers are selected from Drp1 and Drp1-associated proteins. The Drp1-associated proteins comprise proteins that interact with Drp1 directly or indirectly, including but being not limited to, proteins expressed from genes AFG3L2, BCL2, ITPR3, LONP1, OPTN, PHB, and TOMM40; and proteins whose expression are affected by Drp1 overexpression, including but being not limited to, proteins expressed from genes SLC4A7, PDCD2L, UPP1, ARL4C, NELL2, LEF1, SGK1, PDE4D, S1PR3, MCTP2, HES6, and OLR1.

In some embodiments, altered expression of one or more proteins selected from Drp1 and Drp1-associated proteins described herein are indicative of a diagnosis, characterization, or increased risk of death from sepsis. In specific embodiments, upregulated expression of one or more proteins selected from Drp1 and proteins expressed from genes, or example, AFG3L2, BCL2, ITPR3, LONP1, OPTN, PHB, TOMM40, SLC4A7, PDCD2L, ARL4C, NELL2, LEF1, SGK1, S1PR3, HES6, and OLR1 are indicative of a diagnosis, characterization, or increased risk of death from sepsis. In specific embodiments, downregulated expression of one or more proteins selected from proteins expressed from genes, for example, UPP1, PDE4D, and MCTP2 are indicative of a diagnosis, characterization, or increased risk of death from sepsis.

In some embodiments, the present invention provides a gene biomarker for sepsis prognosis, the gene biomarker is preferably selected from Drp1-associated gene signatures. In one embodiment, the Drp1-associated gene signature comprises at least one, two, three, four, five, six, or all of AFG3L2, BCL2, ITPR3, LONP1, OPTN, PHB, and TOMM40. In another embodiment, the Drp1-associated gene signature comprises at least one, two, three, four, five, six, seven, eight, nine, ten, eleven or all of SLC4A7, PDCD2L, UPP1, ARL4C, NELL2, LEF1, SGK1, PDE4D, S1PR3, MCTP2, HES6, and OLR1. In some embodiments, altered expression of one or more genes of the Drp1-associated gene signatures described herein are indicative of a diagnosis, characterization, or increased risk of death from sepsis. In one embodiment, upregulated expression of one or more genes selected from AFG3L2, BCL2, ITPR3, LONP1, OPTN, PHB, TOMM40, SLC4A7, PDCD2L, ARL4C, NELL2, LEF1, SGK1, S1PR3, HES6, and OLR1, and/or downregulated expression of one or more genes selected from UPP1, PDE4D, and MCTP2 are indicative of a diagnosis, characterization, or increased risk of death from sepsis.

In certain embodiments, a sample obtained from a subject is blood (such as whole blood, plasma, serum, or blood cells), tissue (such as skin, liver, or kidney tissues), body fluid (such as saliva, tear, or urine), or a fraction thereof. In some embodiments, the sample is peripheral blood obtained from a subject, and preferably peripheral blood mononuclear cells (PBMCs) separated therefrom. In preferred embodiments, the sample comprises PBMCs. In some embodiments, the sample is subjected to preliminary processing designed to isolate or enrich the sample for the genes or cells that contain the gene. A variety of techniques known to those of ordinary skill in the art may be used for this purpose, including but not limited to, centrifugation; immunocapture, cell lysis, and nucleic acid target captures.

The present invention does not intend to limit the manners to detect the gene expression level, any techniques and methods that are able to test and quantify the expression level of a gene can be applied herein, such as nucleic acid sequencing, nucleic acid hybridization, and nucleic acid amplification. Examples of such techniques and methods include, but are not limited to, polymerase chain reaction (PCR), Quantitative Reverse Transcription Polymerase Chain Reaction (qRT-PCR), transcription-mediated amplification (TMA), ligase chain reaction (LCR), strand displacement amplification (SDA), nucleic acid sequence-based amplification (NASBA), Microarray Analysis, RNA Sequencing (RNA-Seq), Northern Blot, In Situ Hybridization (ISH), Fluorescence In Situ Hybridization (FISH), Single-Cell RNA Sequencing (scRNA-seq), Gene Expression Profiling, Luciferase Assay, Beta-Galactosidase Assay, and Green Fluorescent Protein (GFP) Expression. In one embodiment, the gene expression level can be detected by performing PCR on the sample and detecting amplified nucleic acids in the sample.

In certain embodiments, a variety of nucleic acid sequencing methods are contemplated for use in the methods of the present invention including, for example, next-generation sequencing (NGS), chain terminator (Sanger) sequencing, dye terminator sequencing, nanopore sequencing, single molecule sequencing, and high-throughput sequencing methods.

Non-limiting examples of nucleic acid hybridization techniques include, but are not limited to, in situ hybridization (ISH), microarray, and Southern or Northern blot. In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

Nucleic acids may be amplified prior to or simultaneous with detection. Non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence-based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

In certain embodiments, altered levels of gene expression are detected by altered levels of proteins or polypeptides encoded by the genes (e.g., using immunoassays or mass spectrometry). Illustrative non-limiting examples of immunoassays include, but are not limited to: immunoprecipitation; Western blot; ELISA (Enzyme-Linked Immunosorbent Assay); immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays.

Sepsis Diagnosis and/or Sepsis Prognosis

By virtue of the biomarkers described herein, further developed and provided herein are sepsis diagnostic and/or sepsis prognostic methods that utilize the detection of these biomarkers.

In some embodiments, the present invention provides a method of characterizing sepsis survival risk in a subject. The sepsis survival risk can be characterized by a sepsis survival score, which is calculated as a linear combination of gene expression values and their corresponding weight values in the gene signatures described herein. In such embodiments, the sepsis survival score is calculated using a formula of:

$$sepsis survival score = {\sum }_{i = 1}^{n} {W}_{i} ( \frac{{e}_{i} - {μ}_{i}}{{S}_{i}} ) ,$$

wherein n is the number of genes in the gene signature; Wᵢ is the weighted value of each gene; eᵢ is the expression level of each gene; and μᵢ and Sᵢ are the mean and standard deviation of the gene expression level values, respectively, for the corresponding gene compared to the whole sample or across whole/all samples.

For each gene in the gene signature described herein, μᵢ and Sᵢ can be obtained by detecting the gene expression levels in multiple subjects, and then calculating the mean and standard deviation of the detected gene expression levels. In a specific embodiment, the multiple subjects used for μᵢ and Sᵢ calculation come from a discovery cohort, such as a human peripheral blood mononuclear cell dataset from ArrayExpress, E-MTAB-4421.

For each gene in the gene signature described herein, eᵢ can be obtained by detecting the gene expression level in a sample obtained from a subject. In one embodiment, the subject is one that has been diagnosed with sepsis. In other embodiment, the subject is one that suffers from infections of various degrees although has not been diagnosed with sepsis. In preferred embodiments, the subject is a mammal, preferably a human.

For each gene in the gene signature described herein, Wᵢ depends on how individual gene is weighted among the whole gene signature and can be varied if the gene is weighted differently. In a specific embodiment, Wᵢ is 1 for a gene whose expression is upregulated and is −1 for a gene whose expression is downregulated.

In certain embodiments, the sepsis survival score calculated herein is indicative of the probability of survival from sepsis for a subject, or the probability of septic death for a subject. In some embodiments, a subject with a sepsis survival score that is higher than the mean/medium sepsis survival score is classified as having a low risk of septic death, while a subject with a sepsis survival score that is lower than the mean/medium sepsis survival score is classified as having a high risk of septic death. In a specific embodiment, the mean/medium sepsis survival score is 0.

In one embodiment, the sepsis survival score is calculated based on at least one, two, three, four, five, six, or all genes selected from AFG3L2, BCL2, ITPR3, LONP1, OPTN, PHB, and TOMM40. In another embodiment, the sepsis survival score is calculated based on at least one, two, three, four, five, six, seven, eight, nine, ten, eleven, or all genes selected from SLC4A7, PDCD2L, UPP1, ARL4C, NELL2, LEF1, SGK1, PDE4D, S1PR3, MCTP2, HES6, and OLR1.

In one embodiment, the present invention provides a method of characterizing sepsis survival risk that a subject suffers from, the method comprising the following steps:
(1) obtaining a sample from the subject;
(2) detecting in the sample the expression level of at least one gene selected from the Drp1-associated gene signature described herein; and
(3) calculating a sepsis survival score as a linear combination of gene expression values and their corresponding weight values in the Drp1-associated gene signature described herein.

In such embodiment, the sepsis survival risk that a subject suffers is characterized as the sepsis survival score as calculated above. The characterization of sepsis survival risk can then be applied for various purposes, such as diagnosis, prognosis, and treatment of sepsis.

In one embodiment, step (1) comprises obtaining whole blood, plasma, serum, and/or urine from the subject. In one embodiment, step (1) comprises obtaining peripheral blood from the subject, and optionally separating PBMCs therefrom.

In one embodiment, step (2) comprises detecting in the sample the expression level of at least one, two, three, four, five, or six genes selected from AFG3L2, BCL2, ITPR3, LONP1, OPTN, PHB, and TOMM40. In another embodiment, step (2) comprises detecting in the sample the expression level of at least one, two, three, four, five, six, seven, eight, nine, ten, or eleven genes selected from SLC4A7, PDCD2L, UPP1, ARL4C, NELL2, LEF1, SGK1, PDE4D, S1PR3, MCTP2, HES6, and OLR1. In preferred embodiments, step (2) comprises detecting in the sample the expression levels of genes SLC4A7, PDCD2L, UPP1, ARL4C, NELL2, LEF1, SGK1, PDE4D, S1PR3, MCTP2, HES6, and/or OLR1. In more preferred embodiments, step (2) comprises detecting in the sample the expression levels of genes AFG3L2, BCL2, ITPR3, LONP1, OPTN, PHB, and/or TOMM40.

In one embodiment, step (2) comprises detecting in the sample the expression level of at least one gene selected from the Drp1-associated gene signature described herein by nucleic acid sequencing, nucleic acid hybridization, and/or nucleic acid amplification. In preferred embodiments, step (2) comprises detecting in the sample the expression level of at least one gene selected from the Drp1-associated gene signature described herein by performing PCR on the sample and detecting amplified nucleic acids in the sample.

In one embodiment, step (3) comprises calculating the sepsis survival score using the formula described herein. In one embodiment, step (3) further comprises comparing the calculated sepsis survival score with a mean/medium sepsis survival score, wherein a sepsis survival score that is lower than the mean/medium sepsis survival score is indicative of high sepsis severity in the subject, while a sepsis survival score that is higher than the mean/medium sepsis survival score is indicative of low sepsis severity in the subject. In a specific embodiment, the mean/medium sepsis survival score is 0.

In certain embodiments, lower score values indicate a worse prognosis and survival rate, and thus subjects with lower scores are grouped as the high-risk septic subjects. Subjects with higher scores were classified as low-risk septic subjects. Thus, the method provided by the subject invention can be used to classify a subject to a high-risk or low-risk group.

In certain embodiments, step (3) further comprises comparing the gene expression level(s) as detected in the subject to corresponding reference gene expression level(s). A reference gene expression level is the expression level of a gene in a subject without sepsis or with non-life-threatening sepsis. In preferred embodiments, the reference gene expression level is an average expression level of a gene in more than one subjects without sepsis and/or with non-life-threatening sepsis. In such embodiment, a gene expression level(s) as detected in the subject that is(are) consistent with the corresponding reference gene expression level(s) is indicative of the subject having no sepsis or having non-life threatening sepsis, while a gene expression level(s) as detected in the subject that is(are) inconsistent with the corresponding reference gene expression level(s) is indicative of the subject having sepsis or having life threatening sepsis. In preferred embodiments, a gene expression level(s) as detected in the subject within 1±10% of the corresponding reference gene expression level(s) is(are) indicative of the subject having no sepsis or having non-life threatening sepsis, while a gene expression level(s) as detected in the subject beyond 1±10% of the corresponding reference gene expression level(s) is(are) indicative of the subject having sepsis or having life threatening sepsis.

In some embodiments, the present invention provides a method of determining sepsis severity in a subject, comprising the following steps:
(1) obtaining a sample from the subject;
(2) detecting in the sample the expression level of at least one gene selected from the Drp1-associated gene signature described herein;
(3) calculating a sepsis survival score as a linear combination of gene expression values and their corresponding weight values in the Drp1-associated gene signature described herein; and
(4) determining sepsis severity in the subject according to the sepsis survival score.

In specific embodiments, the subject is one that has been diagnosed with sepsis. In other embodiments, the subject is one that suffers from infections of various degrees although has not been diagnosed with sepsis. In preferred embodiments, the subject is a mammal, preferably a human.

In one embodiment, step (1) comprises obtaining whole blood, plasma, serum, and/or urine from the subject. In one embodiment, step (1) comprises obtaining peripheral blood from the subject, and optionally separating PBMCs therefrom.

In one embodiment, step (2) comprises detecting in the sample the expression level of at least one, two, three, four, five, or six genes selected from AFG3L2, BCL2, ITPR3, LONP1, OPTN, PHB, and TOMM40. In another embodiment, step (2) comprises detecting in the sample the expression level of at least one, two, three, four, five, six, seven, eight, nine, ten, or eleven genes selected from SLC4A7, PDCD2L, UPP1, ARL4C, NELL2, LEF1, SGK1, PDE4D, S1PR3, MCTP2, HES6, and OLR1. In preferred embodiments, step (2) comprises detecting in the sample the expression levels of genes SLC4A7, PDCD2L, UPP1, ARL4C, NELL2, LEF1, SGK1, PDE4D, S1PR3, MCTP2, HES6, and/or OLR1. In preferred embodiments, step (2) further comprises detecting in the sample the expression levels of genes AFG3L2, BCL2, ITPR3, LONP1, OPTN, PHB, and/or TOMM40.

In one embodiment, step (2) comprises detecting in the sample the expression level of at least one gene selected from the Drp1-associated gene signature described herein by nucleic acid sequencing, nucleic acid hybridization, and/or nucleic acid amplification. In preferred embodiments, step (2) comprises detecting in the sample the expression level of at least one gene selected from the Drp1-associated gene signature described herein by performing polymerase chain reaction on the sample and detecting amplified nucleic acids in the sample.

In one embodiment, step (3) comprises calculating the sepsis survival score using the formula described herein.

In one embodiment, step (4) comprises comparing the sepsis survival score calculated in step (3) with a mean/medium sepsis survival score, and determining the subject as having high sepsis severity if the sepsis survival score as calculated is lower than the mean/medium sepsis survival score, while determining the subject as having low sepsis severity if the sepsis survival score as calculated is higher than the mean/medium sepsis survival score. In a specific embodiment, the mean/medium sepsis survival score is 0.

In one embodiment, step (4) comprises determining sepsis severity in the subject solely according to the sepsis survival score calculated by step (3). In another embodiment, step (4) comprises determining sepsis severity in the subject according to a combination of the sepsis survival score calculated in step (3) and other indicators. The other indicators can be ones that are indicative of the progression of sepsis, and/or any other diseases associated with infections of various degrees.

In one embodiment, step (4) further comprises comparing the gene expression level(s) as detected in the subject to corresponding reference gene expression level(s). A reference gene expression level is the expression level of a gene in a subject without sepsis or with non-life-threatening sepsis, or a healthy subject. In preferred embodiments, the reference gene expression level is an average expression level of a gene in more than one subjects without sepsis and/or with non-life-threatening sepsis, or healthy subjects.

In such embodiment, step (4) further comprises determining the subject as having no sepsis or having non-life threatening sepsis if a gene expression level(s) as detected in the subject that is(are) consistent with the corresponding reference gene expression level(s), while determining the subject as having sepsis or having life threatening sepsis if a gene expression level(s) as detected in the subject that is(are) inconsistent with the corresponding reference gene expression level(s). In preferred embodiments, step (4) further comprises determining the subject as having no sepsis or having non-life threatening sepsis if a gene expression level(s) as detected in the subject is(are) within 1±10% of the corresponding reference gene expression level(s), while determining the subject as having sepsis or having life threatening sepsis if a gene expression level(s) as detected in the subject is(are) beyond 1±10% of the corresponding reference gene expression level(s).

In some embodiments, the present invention provides a method of predicting sepsis survival probability for a subject, the method comprising the following steps:
(1) obtaining a sample from the subject;
(2) detecting in the sample the expression level of at least one gene selected from the Drp1-associated gene signature described herein;
(3) calculating a sepsis survival score as a linear combination of gene expression values and their corresponding weight values in the Drp1-associated gene signature described herein; and
(4) predicting sepsis survival probability for the subject according to the sepsis survival score.

In one embodiment, the subject is one that has been diagnosed with sepsis. In other embodiments, the subject is one that suffers from infections of various degrees although has not been diagnosed with sepsis. In preferred embodiments, the subject is a mammal, preferably a human.

In one embodiment, step (1) comprises obtaining whole blood, plasma, serum, and/or urine from the subject. In one embodiment, step (1) comprises obtaining peripheral blood from the subject, and optionally separating PBMCs therefrom.

In one embodiment, step (2) comprises detecting in the sample the expression level of at least one, two, three, four, five, or six genes selected from AFG3L2, BCL2, ITPR3, LONP1, OPTN, PHB, and TOMM40. In another embodiment, step (2) comprises detecting in the sample the expression level of at least one, two, three, four, five, six, seven, eight, nine, ten, or eleven genes selected from SLC4A7, PDCD2L, UPP1, ARL4C, NELL2, LEF1, SGK1, PDE4D, S1PR3, MCTP2, HES6, and OLR1. In preferred embodiments, step (2) comprises detecting in the sample the expression levels of genes SLC4A7, PDCD2L, UPP1, ARL4C, NELL2, LEF1, SGK1, PDE4D, S1PR3, MCTP2, HES6, and/or OLR1. In more preferred embodiments, step (2) comprises detecting in the sample the expression levels of genes AFG3L2, BCL2, ITPR3, LONP1, OPTN, PHB, and/or TOMM40.

In one embodiment, step (2) comprises detecting in the sample the expression level of at least one gene selected from the Drp1-associated gene signature described herein by nucleic acid sequencing, nucleic acid hybridization, and/or nucleic acid amplification. In preferred embodiments, step (2) comprises detecting in the sample the expression level of at least one gene selected from the Drp1-associated gene signature described herein by performing PCR on the sample and detecting amplified nucleic acids in the sample.

In one embodiment, step (3) comprises calculating the sepsis survival score using the formula described herein.

In one embodiment, step (4) comprises comparing the sepsis survival score calculated in step (3) with a mean/medium sepsis survival score, and predicting the subject as having high sepsis survival probability if the sepsis survival score as calculated is higher than the mean/medium sepsis survival score, while predicting the subject as having low sepsis survival probability if the sepsis survival score as calculated is lower than the mean/medium sepsis survival score. In a specific embodiment, the mean/medium sepsis survival score is 0.

In one embodiment, step (4) comprises predicting sepsis survival probability for the subject solely according to the sepsis survival score calculated in step (3). In another embodiment, step (4) comprises predicting sepsis survival probability for the subject according to a combination of the sepsis survival score calculated in step (3) and other indicators. The other indicators can be ones that are indicative of the progression of sepsis, and/or any other diseases associated with infections of various degrees.

In one embodiment, step (4) further comprises comparing the gene expression level(s) as detected in the subject to corresponding reference gene expression level(s). A reference gene expression level is the expression level of a gene in a subject without sepsis or with non-life-threatening sepsis, or a healthy subject. In preferred embodiments, the reference gene expression level is an average expression level of a gene in more than one subjects without sepsis and/or with non-life-threatening sepsis, or healthy subjects.

In such embodiment, step (4) further comprises determining the subject as having no sepsis or having non-life threatening sepsis if a gene expression level(s) as detected in the subject that is(are) consistent with the corresponding reference gene expression level(s), while determining the subject as having sepsis or having life threatening sepsis if a gene expression level(s) as detected in the subject that is(are) inconsistent with the corresponding reference gene expression level(s). In preferred embodiments, step (4) further comprises determining the subject as having no sepsis or having non-life threatening sepsis if a gene expression level(s) as detected in the subject is(are) within 1±10% of the corresponding reference gene expression level(s), while determining the subject as having sepsis or having life threatening sepsis if a gene expression level(s) as detected in the subject is(are) beyond 1±10% of the corresponding reference gene expression level(s).

In some embodiments, the present invention provides a method of predicting septic death probability for a subject, the method comprising the following steps:
(1) obtaining a sample from the subject;
(2) detecting in the sample the expression level of at least one gene selected from the Drp1-associated gene signature described herein;
(3) calculating a sepsis survival score as a linear combination of gene expression values and their corresponding weight values in the Drp1-associated gene signature described herein; and
(4) predicting septic death probability for the subject according to the sepsis survival score.

In one embodiment, the subject is one that has been diagnosed with sepsis. In other embodiments, the subject is one that suffers from infections of various degrees although has not been diagnosed with sepsis. In preferred embodiments, the subject is a mammal, preferably a human.

In one embodiment, step (1) comprises obtaining whole blood, plasma, serum, and/or urine from the subject. In one embodiment, step (1) comprises obtaining peripheral blood from the subject, and optionally separating PBMCs therefrom.

In one embodiment, step (2) comprises detecting in the sample the expression level of at least one, two, three, four, five, or six genes selected from AFG3L2, BCL2, ITPR3, LONP1, OPTN, PHB, and TOMM40. In another embodiment, step (2) comprises detecting in the sample the expression level of at least one, two, three, four, five, six, seven, eight, nine, ten, or eleven genes selected from SLC4A7, PDCD2L, UPP1, ARL4C, NELL2, LEF1, SGK1, PDE4D, S1PR3, MCTP2, HES6, and OLR1. In preferred embodiments, step (2) comprises detecting in the sample the expression levels of genes SLC4A7, PDCD2L, UPP1, ARL4C, NELL2, LEF1, SGK1, PDE4D, S1PR3, MCTP2, HES6, and/or OLR1. In more preferred embodiments, step (2) comprises detecting in the sample the expression levels of genes AFG3L2, BCL2, ITPR3, LONP1, OPTN, PHB, and/or TOMM40.

In one embodiment, step (2) comprises detecting in the sample the expression level of at least one gene selected from the Drp1-associated gene signature described herein by nucleic acid sequencing, nucleic acid hybridization, and/or nucleic acid amplification. In preferred embodiments, step (2) comprises detecting in the sample the expression level of at least one gene selected from the Drp1-associated gene signature described herein by performing PCR on the sample and detecting amplified nucleic acids in the sample.

In one embodiment, step (3) comprises calculating the sepsis survival score using the formula described herein.

In one embodiment, step (4) comprises comparing the sepsis survival score calculated in step (3) with a mean/medium sepsis survival score, and predicting the subject as having a high septic death probability if the sepsis survival score as calculated is lower than the mean/medium sepsis survival score, while predicting the subject as having a low septic death probability if the sepsis survival score as calculated is higher than the mean/medium sepsis survival score. In a specific embodiment, the mean/medium sepsis survival score is 0.

In one embodiment, step (4) comprises predicting septic death probability for the subject solely according to the sepsis survival score calculated in step (3). In another embodiment, step (4) comprises predicting septic death probability for the subject according to a combination of the sepsis survival score calculated in step (3) and other indicators. The other indicators can be ones that are indicative of the progression of sepsis, and/or any other diseases associated with infections of various degrees.

In one embodiment, step (4) further comprises comparing the gene expression level(s) as detected in the subject to corresponding reference gene expression level(s). A reference gene expression level is the expression level of a gene in a subject without sepsis or with non-life-threatening sepsis, a healthy subject. In preferred embodiments, the reference gene expression level is an average expression level of a gene in more than one subjects without sepsis and/or with non-life-threatening sepsis, or healthy subjects.

In such embodiment, step (4) further comprises determining the subject as having no sepsis or having non-life-threatening sepsis if a gene expression level(s) as detected in the subject that is(are) consistent with the corresponding reference gene expression level(s), while determining the subject as having sepsis or having life threatening sepsis if a gene expression level(s) as detected in the subject that is(are) inconsistent with the corresponding reference gene expression level(s). In preferred embodiments, step (4) further comprises determining the subject as having no sepsis or having non-life-threatening sepsis if a gene expression level(s) as detected in the subject is(are) within 1±10% of the corresponding reference gene expression level(s), while determining the subject as having sepsis or having life threatening sepsis if a gene expression level(s) as detected in the subject is(are) beyond 1±10% of the corresponding reference gene expression level(s).

In certain embodiments, the method of the subject invention further comprises:
(5) treating the subject for sepsis;
(6) obtain a sample from the subject after the treatment for sepsis;
(7) determining in the sample obtained after treatment for sepsis the expression level of at least one gene selected from the Drp1-associated gene signature including Drp1-DEGs gene signature and/or Drp1-PPI gene signature, wherein the determination comprises detecting in the sample the expression level of at least one, two, three, four, five, or six genes selected from AFG3L2, BCL2, ITPR3, LONP1, OPTN, PHB, and TOMM40, and/or detecting in the sample the expression level of at least one, two, three, four, five, six, seven, eight, nine, ten, or eleven genes selected from SLC4A7, PDCD2L, UPP1, ARL4C, NELL2, LEF1, SGK1, PDE4D, S1PR3, MCTP2, HES6, and OLR1;
(8) calculating a sepsis survival score as a linear combination of gene expression values and their corresponding weight values in the Drp1-associated gene signature described herein after treatment for sepsis; and
(9) determining the treatment as a favorable treatment that reduces septic death probability if the sepsis survival score increases after the treatment, or determining the treatment as an unfavorable treatment that fails to reduce septic death probability if the sepsis survival score remains unchanged or decreases after the treatment; or
determining the treatment as a favorable treatment that increases sepsis survival probability if the sepsis survival score increases after the treatment, or determining the treatment as an unfavorable treatment that fails to increase sepsis survival probability if the sepsis survival score remains unchanged or decreases after the treatment; or
determining the treatment as a favorable treatment that improves the condition of the subject or reduces sepsis severity if the sepsis survival score increases after the treatment, or determining the treatment as an unfavorable treatment that fails to improve the condition of the subject or reduce sepsis severity if the sepsis survival score remains unchanged or decreases after the treatment; or
determining the treatment as a favorable treatment such that the subject can be classified as a low-risk septic subject if the sepsis survival score increases after the treatment, or determining the treatment as a unfavorable treatment such that the subject remains in the same classified group of a low-risk or high-risk septic subject if the sepsis survival score remains unchanged or decreases after the treatment.

In one embodiment, the subject invention provides a method of reducing sepsis severity in a subject, the method comprising the following steps:
obtaining a sample (e.g., blood, tissues or body fluid) from the subject;
detecting in the sample the expression level of at least one gene selected from at least one Drp1-associated gene signature;
calculating a first sepsis survival score as a linear combination of gene expression values and their corresponding weight values in the at least one Drp1-associated gene signature;
treating the subject for sepsis;
obtaining a sample (e.g., blood, tissues or body fluid) from the subject after the treatment;
detecting in the sample after the treatment the expression level of at least one gene selected from at least one Drp1-associated gene signature;
calculating a second sepsis survival score as a linear combination of gene expression values and their corresponding weight values in the at least one Drp1-associated gene signature;
determining a reduced sepsis severity in the subject if the second sepsis survival score is higher than the first sepsis survival score or determining a maintained or worsen sepsis severity in the subject if the second sepsis survival score is equal to or lower than the first sepsis survival score.

In preferred embodiments, the at least one Drp1-associated gene signature is Drp1-DGEs gene signature and/or Drp1-PPI gene signature, wherein the Drp1-PPI gene signature comprised AFG3L2, BCL2, ITPR3, LONP1, OPTN, PHB, and TOMM40, and the Drp1-DGEs gene signature comprises SLC4A7, PDCD2L, UPP1, ARL4C, NELL2, LEF1, SGK1, PDE4D, S1PR3, MCTP2, HES6, and OLR1.

In a specific embodiment, the at least one gene is selected from AFG3L2, BCL2, ITPR3, LONP1, OPTN, PHB, TOMM40, SLC4A7, PDCD2L, UPP1, ARL4C, NELL2, LEF1, SGK1, PDE4D, S1PR3, MCTP2, HES6, and OLR1.

In certain embodiments, the method further comprises comparing the first and/or second sepsis survival score with a mean/medium sepsis survival score, and determining the subject as with high sepsis severity if the first and/or second sepsis survival score is lower than the mean/medium sepsis survival score, while determining the subject as with low sepsis severity if the first and/or second sepsis survival score is higher than the mean/medium sepsis survival score. In a specific embodiment, the mean/medium sepsis survival score is 0.

In specific embodiments, the samples obtained before and after the treatment are of the same type. In specific embodiments, the at least one gene detected for the expression levels before and after the treatment are the same group of genes.

In certain embodiments, the method of the subject invention further comprises detecting/measuring the expression level of Drp1 in the sample of the subject and/or detecting/measuring the mitochondrial function. In a preferred embodiment, Drp1 is overexpressed in the sample of the subject.

### Sepsis Therapies

In some embodiments, the present invention provides a method of determining efficacy of a therapy for sepsis in a subject in need thereof, the method comprising the following steps:
(1) obtaining a sample from the subject, detecting in the sample the expression level of at least one gene selected from the Drp1-associated gene signature described herein, and calculating a sepsis survival score (e.g., a first sepsis survival score) as a linear combination of gene expression values and their corresponding weight values in the Drp1-associated gene signature described herein;
(2) administering the therapy to the subject;
(3) obtaining a sample from the subject after the therapy, detecting in the sample obtained after the therapy the expression level of at least one gene selected from the Drp1-associated gene signature described herein, and calculating a sepsis survival score after the therapy (e.g., a second first sepsis survival score) using the formula described herein; and
(4) determining efficacy of the therapy for sepsis in the subject.

In one embodiment, the subject is one that has been diagnosed with sepsis. In other embodiments, the subject is one that suffers from infections of various degrees although has not been diagnosed with sepsis. In preferred embodiments, the subject is a mammal, preferably a human.

The therapy can be various treatments and interventions used to manage, alleviate, or cure sepsis or sepsis-associated symptoms, including but being not limited to, medications, chemotherapy, biologics, surgery, radiation therapy, physical therapy, occupational therapy, speech therapy, psychological therapy, acupuncture, herbal medicine, acupuncture, mind-body therapy, cardiac rehabilitation, pulmonary rehabilitation, neurological rehabilitation, gene therapy, such as gene editing and gene replacement, cell therapy, such as stem cell therapy, immunotherapy, and any combination thereof.

In one embodiment, the therapy is an anti-inflammatory therapy, such as, by administration of one or more antibiotics and/or corticosteroids. In one embodiment, the therapy is an immunomodulatory therapy, such as, by administration of one or more immunomodulators. In one embodiment, the therapy is a fluid resuscitation therapy, such as, by administration of one or more vasopressors.

In one embodiment, step (2) comprises administering one or more of antibiotics, corticosteroids, immunomodulators, and vasopressors to the subject.

In one embodiment, step (1) and/or step (3) comprises obtaining whole blood, plasma, serum, and/or urine from the subject. In one embodiment, the samples obtained in step (1) and step (3) are of the same type. In another embodiment, the samples obtained in step (1) and step (3) are of different types. In a specific embodiment, both step (1) and step (3) comprise obtaining peripheral blood from the subject, and optionally separating PBMCs therefrom.

In one embodiment, the genes whose expression levels are detected in step (1) and step (3) are the same group of genes. In one embodiment, both step (1) and step (3) comprise detecting in the sample the expression level of at least one, two, three, four, five, or six genes selected from AFG3L2, BCL2, ITPR3, LONP1, OPTN, PHB, and TOMM40. In another embodiment, both step (1) and step (3) comprise detecting in the sample the expression level of at least one, two, three, four, five, six, seven, eight, nine, ten, or eleven genes selected from SLC4A7, PDCD2L, UPP1, ARL4C, NELL2, LEF1, SGK1, PDE4D, S1PR3, MCTP2, HES6, and OLR1. In preferred embodiments, both step (1) and step (3) comprise detecting in the sample the expression levels of genes SLC4A7, PDCD2L, UPP1, ARL4C, NELL2, LEF1, SGK1, PDE4D, S1PR3, MCTP2, HES6, and/or OLR1. In more preferred embodiments, both step (1) and step (3) comprise detecting in the sample the expression levels of genes AFG3L2, BCL2, ITPR3, LONP1, OPTN, PHB, and/or TOMM40.

In one embodiment, step (1) and/or step (3) comprise detecting in the sample the expression level of at least one gene selected from the Drp1-associated gene signature described herein by nucleic acid sequencing, nucleic acid hybridization, and/or nucleic acid amplification. In preferred embodiments, step (1) and/or step (3) comprise detecting in the sample the expression level of at least one gene selected from the Drp1-associated gene signature described herein by performing PCR on the sample and detecting amplified nucleic acids in the sample.

In one embodiment, step (1) and/or step (3) comprise calculating the sepsis survival score using the formula described herein.

In one embodiment, step (1) can further comprise comparing the gene expression level(s) as detected in the subject to corresponding reference gene expression level(s). A reference gene expression level is the expression level of a gene in a subject without sepsis or with non-life-threatening sepsis, or a healthy subject. In preferred embodiments, the reference gene expression level is an average expression level of a gene in more than one subjects without sepsis and/or with non-life-threatening sepsis, or healthy subjects.

In one embodiment, step (1) can further comprise comparing the sepsis survival score calculated in step (1) with a mean/medium sepsis survival score, and determining the subject as with high sepsis severity if the sepsis survival score as calculated is lower than the mean/medium sepsis survival score, while determining the subject as with low sepsis severity if the sepsis survival score as calculated is higher than the mean/medium sepsis survival score. In a specific embodiment, the mean/medium sepsis survival score is 0.

In one embodiment, step (4) comprises comparing the sepsis survival score calculated in step (3) with the sepsis survival score calculated in step (1), and determining the therapy as being effective for sepsis in the subject if the sepsis survival score calculated in step (3) is higher than the sepsis survival score calculated in step (1), while determining the therapy as being ineffective for sepsis in the subject if the sepsis survival score calculated in step (3) is equal to or lower than the sepsis survival score calculated in step (1).

In one embodiment, step (4) comprises determining efficacy of the therapy for sepsis in the subject solely according to a comparison between the sepsis survival scores calculated in steps (1) and (3). In another embodiment, step (4) comprises determining efficacy of the therapy for sepsis in the subject according to a combination of a comparison between the sepsis survival scores calculated in steps (1) and (3), and other indicators. The other indicators can be ones that are indicative of the progression of sepsis, and/or any other diseases associated with infections of various degrees.

In one embodiment, step (4) further comprises comparing the gene expression level(s) as detected in the subject to corresponding reference gene expression level(s). A reference gene expression level is the expression level of a gene in a subject without sepsis or with non-life-threatening sepsis, or a healthy subject. In preferred embodiments, the reference gene expression level is an average expression level of a gene in more than one subject without sepsis and/or with non-life-threatening sepsis, or healthy subjects.

In such embodiment, step (4) further comprises determining the therapy as being effective for sepsis in the subject if the gene expression level(s) as detected in the subject is(are) consistent with the corresponding reference gene expression level(s), while determining the therapy as being ineffective for sepsis in the subject if the gene expression level(s) as detected in the subject is(are) inconsistent with the corresponding reference gene expression level(s). In preferred embodiments, step (4) further comprises determining the therapy as being effective for sepsis in the subject if the gene expression level(s) as detected in the subject is(are) within 1±10% of the corresponding reference gene expression level(s), while determining the therapy as being ineffective for sepsis in the subject if the gene expression level(s) as detected in the subject is(are) beyond 1±10% of the corresponding reference gene expression level(s).

In some embodiments, the present invention provides a method of treating sepsis in a subject in need thereof, the method comprising the following steps:
(1) administering a therapy for sepsis to the subject;
(2) at any point of the sepsis treatment, obtaining a sample from the subject, detecting in the sample the expression level of at least one gene selected from the Drp1-associated gene signature described herein, and calculating a sepsis survival score as a linear combination of gene expression values and their corresponding weight values in the Drp1-associated gene signature described herein; and
(3) modulating treatment strategy according to alteration of the sepsis survival score.

In one embodiment, the subject is one that has been diagnosed with sepsis. In other embodiments, the subject is one that suffers from infections of various degrees although has not been diagnosed with sepsis. In preferred embodiments, the subject is a mammal, preferably a human.

The therapy can be various treatments and interventions used to manage, alleviate, or cure sepsis or sepsis-associated symptoms, including but being not limited to, medications, chemotherapy, biologics, surgery, radiation therapy, physical therapy, occupational therapy, speech therapy, psychological therapy, acupuncture, herbal medicine, acupuncture, mind-body therapy, cardiac rehabilitation, pulmonary rehabilitation, neurological rehabilitation, gene therapy, such as gene editing and gene replacement, cell therapy, such as stem cell therapy, immunotherapy, and any combination thereof.

In one embodiment, the therapy is an anti-inflammatory therapy, such as, by administration of one or more antibiotics and/or corticosteroids. In one embodiment, the therapy is an immunomodulatory therapy, such as, by administration of one or more immunomodulators. In one embodiment, the therapy is a fluid resuscitation therapy, such as, by administration of one or more vasopressors.

In specific embodiments, step (1) comprises administering one or more of antibiotics, corticosteroids, immunomodulators, and vasopressors to the subject.

In certain embodiments, step (2) can be performed at any point before, during, or after the administration of the therapy for sepsis. In some embodiments, step (2) is performed regularly, such as once per day, or once per week. In a specific embodiment, step (2) is performed before and after each administration of treatment. In some embodiments, step (2) is performed repeatedly at least twice, at least three times, at least four times, at least five times, or at least six times throughout the sepsis treatment.

In one embodiment, step (2) comprises obtaining whole blood, plasma, serum, and/or urine from the subject. In one embodiment, the samples obtained in step (2) are of the same type for each repetition of step (2) in the subject. In another embodiment, the samples obtained in step (2) are of different types for each repetition of step (2) in the subject. In one embodiment, step (2) comprises obtaining peripheral blood from the subject, and optionally separating PBMCs therefrom.

In one embodiment, the genes whose expression levels are detected in step (2) are the same group of genes for each repetition of step (2) in the subject. In one embodiment, step (2) comprises detecting in the sample the expression level of at least one, two, three, four, five, or six genes selected from AFG3L2, BCL2, ITPR3, LONP1, OPTN, PHB, and TOMM40. In another embodiment, step (2) comprises detecting in the sample the expression level of at least one, two, three, four, five, six, seven, eight, nine, ten, or eleven genes selected from SLC4A7, PDCD2L, UPP1, ARL4C, NELL2, LEF1, SGK1, PDE4D, S1PR3, MCTP2, HES6, and OLR1. In preferred embodiments, step (2) comprises detecting in the sample the expression levels of genes SLC4A7, PDCD2L, UPP1, ARL4C, NELL2, LEF1, SGK1, PDE4D, S1PR3, MCTP2, HES6, and/or OLR1. In more preferred embodiments, step (2) comprises detecting in the sample the expression levels of genes AFG3L2, BCL2, ITPR3, LONP1, OPTN, PHB, and/or TOMM40.

In one embodiment, step (2) comprises detecting in the sample the expression level of at least one gene selected from the Drp1-associated gene signature described herein by nucleic acid sequencing, nucleic acid hybridization, and/or nucleic acid amplification. In preferred embodiments, step (2) comprises detecting in the sample the expression level of at least one gene selected from the Drp1-associated gene signature described herein by performing PCR on the sample and detecting amplified nucleic acids in the sample.

In one embodiment, step (2) comprises calculating a sepsis survival score using the formula described herein. In some embodiment, each repetition of step (2) comprises calculating a sepsis survival score in step (2) using the formula described herein.

In one embodiment, step (3) comprises strengthening, maintaining, attenuating, or changing treatment strategy according to the alteration of the sepsis survival score(s). In a specific embodiment, step (3) comprises strengthening or changing treatment strategy, such as increasing administration dosage and/or frequency, or changing to a new therapy, if the sepsis survival score decreases or keeps unchanged among at least two repetitions of step (2); while maintaining or attenuating treatment strategy, such as decreasing administration dosage and/or frequency, if the sepsis survival score increases among at least two repetitions of step (2).

In certain embodiments, step (2) further comprises comparing the sepsis survival score calculated in step (2) with a mean/medium sepsis survival score, and determining the subject as with high sepsis severity if the sepsis survival score as calculated is lower than the mean/medium sepsis survival score, while determining the subject as with low sepsis severity if the sepsis survival score as calculated is higher than the mean/medium sepsis survival score. In a specific embodiment, the mean/medium sepsis survival score is 0.

In certain embodiments, each sepsis survival score calculated in each repetition of step (2) is compared to a mean/medium sepsis survival score. As such, the sepsis severity of the subject can be determined and monitored throughout the treatment for sepsis.

In certain embodiments, step (3) can further comprise strengthening or changing treatment strategy, such as increasing administration dosage and/or frequency, or changing to a new therapy, if the sepsis survival score as calculated is lower than the mean/medium sepsis survival score in at least one repetition of step (2); while maintaining or attenuating treatment strategy, such as decreasing administration dosage and/or frequency, if the sepsis survival score as calculated is higher than the mean/medium sepsis survival score in at least one repetition of step (2).

In certain embodiments, step (2) can further comprise comparing the gene expression level(s) as detected in the subject to corresponding reference gene expression level(s). A reference gene expression level is the expression level of a gene in a subject without sepsis or with non-life-threatening sepsis, or a healthy subject. In preferred embodiments, the reference gene expression level is an average expression level of a gene in more than one subject without sepsis and/or with non-life-threatening sepsis, or healthy subjects.

In some embodiments, step (3) can further comprise strengthening or changing treatment strategy, such as increasing administration dosage and/or frequency, or changing to a new therapy, if the gene expression level(s) as detected in the subject is(are) inconsistent with the corresponding reference gene expression level(s) in at least one repetition of step (2), while maintaining or attenuating treatment strategy, such as decreasing administration dosage and/or frequency, if the gene expression level(s) as detected in the subject is(are) consistent with the corresponding reference gene expression level(s) in at least one repetition of step (2). In preferred embodiments, step (3) further comprises maintaining or attenuating treatment strategy, such as decreasing administration dosage and/or frequency, if the gene expression level(s) as detected in the subject is(are) within 1±10% of the corresponding reference gene expression level(s) in at least one repetition of step (2), while strengthening or changing treatment strategy, such as increasing administration dosage and/or frequency, or changing to a new therapy, if the gene expression level(s) as detected in the subject is(are) beyond 1±10% of the corresponding reference gene expression level(s) in at least one repetition of step (2).

In some embodiments, the present invention provides a method of treating sepsis in a subject in need thereof, the method comprising the following steps:
(1) obtaining a sample from the subject;
(2) detecting in the sample the expression level of at least one gene selected from the Drp1-associated gene signature described herein;
(3) calculating a sepsis survival score as a linear combination of gene expression values and their corresponding weight values in the Drp1-associated gene signature described herein; and
(4) treating the subject according to the sepsis survival score.

In one embodiment, the subject is one that has been diagnosed with sepsis. In other embodiments, the subject is one that suffers from infections of various degrees although has not been diagnosed with sepsis. In preferred embodiments, the subject is a mammal, preferably a human.

In specific embodiments, step (1) comprises obtaining whole blood, plasma, serum, and/or urine from the subject. In a specific embodiment, step (1) comprises obtaining peripheral blood from the subject, and optionally separating PBMCs therefrom.

In one embodiment, step (2) comprises detecting in the sample the expression level of at least one, two, three, four, five, or six genes selected from AFG3L2, BCL2, ITPR3, LONP1, OPTN, PHB, and TOMM40. In another embodiment, step (2) comprises detecting in the sample the expression level of at least one, two, three, four, five, six, seven, eight, nine, ten, or eleven genes selected from SLC4A7, PDCD2L, UPP1, ARL4C, NELL2, LEF1, SGK1, PDE4D, S1PR3, MCTP2, HES6, and OLR1. In preferred embodiments, step (2) comprises detecting in the sample the expression levels of genes SLC4A7, PDCD2L, UPP1, ARL4C, NELL2, LEF1, SGK1, PDE4D, S1PR3, MCTP2, HES6, and/or OLR1. In more preferred embodiments, step (2) comprises detecting in the sample the expression levels of genes AFG3L2, BCL2, ITPR3, LONP1, OPTN, PHB, and/or TOMM40.

In one embodiment, step (2) comprises detecting in the sample the expression level of at least one gene selected from the Drp1-associated gene signature described herein by nucleic acid sequencing, nucleic acid hybridization, and/or nucleic acid amplification. In preferred embodiments, step (2) comprises detecting in the sample the expression level of at least one gene selected from the Drp1-associated gene signature described herein by performing PCR on the sample and detecting amplified nucleic acids in the sample.

In one embodiment, step (3) comprises calculating the sepsis survival score using the formula described herein.

In one embodiment, step (4) comprises comparing the sepsis survival score calculated in step (3) with a mean/medium sepsis survival score, and treating the subject with a sepsis therapy, for example, treating the subject by decreasing levels of neutrophils and/or increasing activation of NK cells if the sepsis survival score as calculated is lower than the mean/medium sepsis survival score. In a specific embodiment, the mean/medium sepsis survival score is 0.

In one embodiment, step (4) can further comprise comparing the gene expression level(s) as detected in the subject to corresponding reference gene expression level(s). A reference gene expression level is the expression level of a gene in a subject without sepsis or with non-life-threatening sepsis, or a healthy subject. In preferred embodiments, the reference gene expression level is an average expression level of a gene in more than one subject without sepsis and/or with non-life-threatening sepsis, or an average expression level of a gene in more than one healthy subject.

In specific embodiments, step (4) can further comprise treating the subject by decreasing levels of neutrophils and/or increasing activation of NK cells if the gene expression level(s) as detected in the subject is(are) inconsistent with the corresponding reference gene expression level(s). In preferred embodiments, step (4) further comprises treating the subject by decreasing levels of neutrophils and/or increasing activation of NK cells if the gene expression level(s) as detected in the subject is(are) beyond 1±10% of the corresponding reference gene expression level(s).

In one embodiment, the present invention provides a method of treating sepsis in a subject in need thereof, the method comprising the following steps:
(1) obtaining a sample (e.g., blood, tissues or body fluid) from the subject;
(2) detecting in the sample the expression level of at least one gene selected from the Drp1-associated gene signature including Drp1-DGEs gene signature and Drp1-PPI gene signature;
(3) calculating a first sepsis survival score as a linear combination of gene expression values and their corresponding weight values of the at least one gene selected from the Drp1-associated gene signature;
(4) treating the subject for sepsis;
(5) obtaining a sample (e.g., blood, tissues or body fluid) from the subject after treatment for sepsis;
(6) detecting in the sample after the treatment the expression level of at least one gene selected from the Drp1-associated gene signature including Drp1-DGEs gene signature and Drp1-PPI gene signature;
(7) calculating a second sepsis survival score as a linear combination of gene expression values and their corresponding weight values of the at least one gene selected from the Drp1-associated gene signature; and
(8) determining the treatment as a favorable or effective treatment if the sepsis survival score increases after the treatment, or determining the treatment as an unfavorable or ineffective treatment if the sepsis survival score remains unchanged or decreases after the treatment; or
determining the treatment as a favorable or effective treatment if the second sepsis survival score is higher than the first sepsis survival score, or determining the treatment as an unfavorable or ineffective treatment if the second sepsis survival score is equal to or lower than the first sepsis survival score.

In preferred embodiments, step (2) comprises detecting in the sample the expression level of at least one gene selected from AFG3L2, BCL2, ITPR3, LONP1, OPTN, PHB, and TOMM40, and/or detecting in the sample the expression level of at least one gene selected from SLC4A7, PDCD2L, UPP1, ARL4C, NELL2, LEF1, SGK1, PDE4D, S1PR3, MCTP2, HES6, and OLR1.

In preferred embodiments, step (6) comprises detecting in the sample the expression level of at least one gene selected from AFG3L2, BCL2, ITPR3, LONP1, OPTN, PHB, and TOMM40, and/or detecting in the sample the expression level of at least one gene selected from SLC4A7, PDCD2L, UPP1, ARL4C, NELL2, LEF1, SGK1, PDE4D, S1PR3, MCTP2, HES6, and OLR1.

In specific embodiments, the at least one gene is selected from AFG3L2, BCL2, ITPR3, LONP1, OPTN, PHB, TOMM40, SLC4A7, PDCD2L, UPP1, ARL4C, NELL2, LEF1, SGK1, PDE4D, S1PR3, MCTP2, HES6, and OLR1.

In specific embodiments, the samples obtained before and after the treatment are of the same type. In specific embodiments, the at least one gene detected for the expression levels before and after the treatment are the same group of genes.

In certain embodiments, the method of the subject invention further comprises the steps of obtaining a sample from a healthy subject; detecting the expression level of at least one gene selected from the Drp1-associated gene signature including Drp1-DGEs gene signature and Drp1-PPI gene signature; and calculating a sepsis survival score as a linear combination of gene expression values and their corresponding weight values of the Drp1-associated gene signature described herein.

In certain embodiments, the method of the subject invention further comprises a step of comparing each of the calculated the sepsis survival score, as described in the subject invention, from the subject having sepsis to a sepsis survival score calculated from the healthy subject, which may also be indicative of whether a treatment of sepsis is a effective or favorable treatment, or whether the severity of sepsis in the subject has improved or not, or whether the subject having sepsis has a higher chance of surviving.

In certain embodiments, the method of the subject invention further comprises detecting/measuring the expression level of Drp1 in the sample of the subject and/or detecting/measuring the mitochondrial function. In a preferred embodiment, Drp1 is overexpressed in the sample of the subject.

In certain embodiments, the method of the subject invention may also be used to treat a condition associated with sepsis, which includes, but is not limited to, for example, severe sepsis, septic shock, multiple organ dysfunction syndrome (MODS) and systemic inflammatory response syndrome (SIRS) when caused by infection. In particular, severe sepsis septic shock MODS and SIRS are typically associated to sepsis in process that usually begins with infection, and sepsis and results in organ dysfunction.

In certain embodiments, the therapy is an anti-inflammatory therapy, such as, by administration of one or more antibiotics and/or corticosteroids. In some embodiments the one or more antibiotics can be administered simultaneously, or sequentially, one with respect to the other and with respect to the one or more corticosteroids. In some embodiments, exemplary antibiotics include, but are not limited to, vancomycin, ceftriaxone (Rocephin™), ceftazidime (Fortaz™), cefepime (Maxipime™), cefotaxime (Claforan™), clindamycin (Cleocin™), imipenem/cilastatin (Primaxin™), levofloxacin (Levaquin™), meropenem (Merrem™), ampicillin and sulbactam (Unasyn), piperacillin and tazobactam (Zosyn™).

In one embodiment, the therapy is an immunomodulatory therapy, such as, by administration of one or more immunomodulators. Exemplary immunomodulators include, but are not limited to, cytokines (e.g., interleukin-2 (IL-2)), interferons, such as interferon (IFN), and corticosteroids, such as cortisone, hydrocortisone, methylprednisolone, betamethasone, and dexamethasone. In one embodiment, the therapy is a fluid resuscitation therapy, such as, by administration of one or more vasopressors.

In one embodiment, the treatment and/or therapy is administered to the subject by standard routes. In certain embodiments, the administration according to the subject invention includes, but is not limited to, for example, oral, rectal, bronchial, nasal, topical, buccal, ophthalmic, sub-lingual, transdermal, vaginal, intra-arterial, parenteral (e.g., intravenous, intraperitoneal, intradermal, subcutaneous or intramuscular), intracranial, intracerebral, intrauterine, intraspinal, interocular administration. In certain embodiments, the administration is systemic administration, which can be by transmucosal or transdermal means. For transmucosal or transdermal administration.

In some embodiments, administration may be also by way of other carriers or vehicles such as patches, micelles, liposomes, vesicles, implants (e.g., microimplants), synthetic polymers, microspheres, nanoparticles, and the like. Depending on the condition being treated, one route may be preferred over others, which can be determined by those skilled in the art.

### EXAMPLES

### Materials and Methods

### Microarray Datasets and Sepsis Survival-Related Genes

Genes that are differentially expressed between patients with a low or high risk of sepsis-related death have been previously identified by Feng, A. et al. (Feng, A. et al., NAMPT-associated gene signature in the prediction of severe sepsis. Am J Transl Res 2022, 14, (10), 7090-7097). These genes were identified using two human peripheral blood mononuclear cell datasets from ArrayExpress, E-MTAB-4421 and E-MTAB-4451. Samples from these datasets were collected from patients at 4 weeks after intensive care unit admission. Differentially expressed genes (DEGs) were only considered significant if the fold change was greater than 1.5 and the false discovery rate (FDR) was less than 5%. In the present invention, E-MTAB-4421 was used as the discovery cohort and E-MTAB-4451 was used as the validation cohort to test this Drp1-gene signature.

### Sepsis Survival Score

To generate a practical sepsis survival score, a linear combination of gene expression values and their corresponding weight values in the Drp1-associated gene signature was used. The sepsis survival score is as follows:

$$sepsis survival score = {\sum }_{i = 1}^{n} {W}_{i} ( \frac{{e}_{i} - {μ}_{i}}{{S}_{i}} )$$

Wherein n is the number of genes in the Drp1-associated gene signature for each dataset, Wᵢ shows the weighted value of each gene, eᵢ is the expression level of each gene, and μᵢ and Sᵢ are the mean and standard deviation values, respectively, for the corresponding gene compared the whole sample. Scores above the mean/medium sepsis survival score were classified into the high sepsis survival score cohort, while the values below the mean were classified into the low sepsis survival score cohort.

### Enrichment Analysis

To generate a list of Drp1-related genes, genes that significantly changed during Drp1 upregulation was first identified using the Gene Expression Omnibus (GEO) dataset GSE182710, referred to as differentially expressed genes (DEGs). To be classified as a DEG, the genes met the following criteria: fold change >1.5, FDR<0.05. Here, these genes were referred to as the DEGs-based Drp1-associated genes. Next, a protein-protein interaction (PPI) analysis of the protein, Drp1, was performed to identify proteins that are predicted to interact with Drp1 using the STRING database. The STRING database identifies functional protein association networks. In the analysis, only the primary protein interactors (also known as 1^{st} shell interactors) were used, which are proteins that are directly associated with Drp1. Proteins with interaction scores greater than 0.7 were selected and henceforth referred to as PPI-based Drp1-associated genes. The protein network was visualized by Cytoscape (3.10.2).

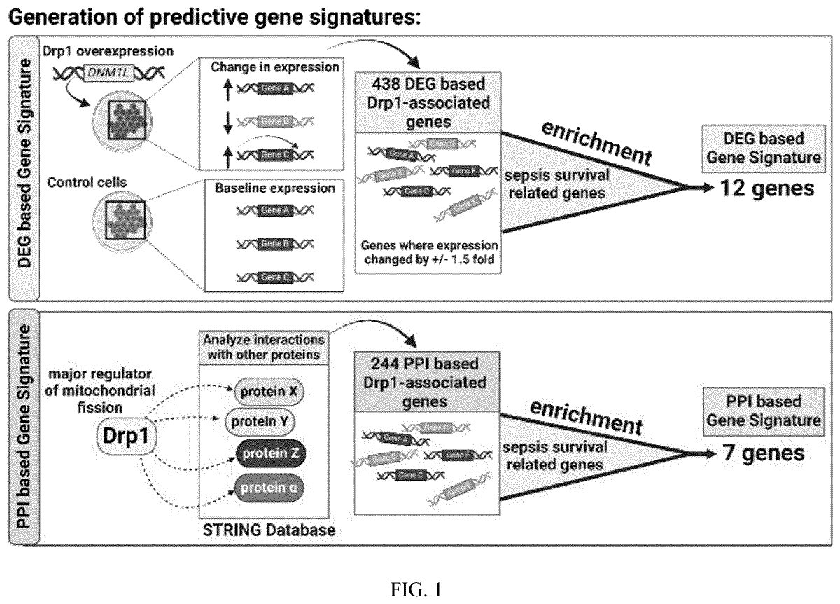

The two Drp1-associated gene signatures (GS) were obtained by identifying the common genes between sepsis survival DEGs and the two Drp1-associated genes described above (DEGs-based and PPI-based, FIG. 1). This enrichment resulted in a 12-gene DEG-based GS and 7-gene PPI-based GS.

Next, Kyoto Encyclopedia of Genes and Genomes (KEGG) analysis of the generated Drp1-associated genes were performed using the database for annotation, visualization, and integrated discovery (DAVID, version 6.8, david.ncifcrf.gov/tools.jsp). DAVID is a biological and functional annotation database that aids in the interpretation of gene/signaling pathway interactions. KEGG analysis was repeated for the sepsis-survival genes. Irrelevant pathways, such as diseases that are not associated with sepsis, were excluded. Adjusted p-values less than 0.05 were considered to be significant.

To validate the accuracy of the PPI analysis, correlation matrixes were generated for both GS using the R package “corrplot” (version 0.92). The genomic annotation file for humans (Genome assembly GRCh37) was obtained from the UCSC Genome Browser. The circular plot was generated by the R package “circlize” (version 0.4.10). All sepsis survival-related genes were visualized as dots, and genes in two gene signatures were marked in the inner circle.

### Evaluating Prognostic Capabilities of GS

To validate that these GSs have the prognostic capability of distinguishing between high and low sepsis risk groups, a variety of assessments were performed. First, gene expression heatmaps of both GS were generated and patients were grouped by their sepsis-risk group. The heatmaps were visualized using the R package “gplots” (version 3.1.3.1). The method “complete” was used for hierarchical clustering, and the method “Euclidean” was utilized for distance calculation. The survival scores for each GS were calculated and then plotted in violin plots using the R package “ggplot2” (version 3.5.1). The ROC curves were generated by R package “PROC” (version 1.18.5), and the AUC values were calculated by the function “auc”.

### Running Enrichment Score

To determine the running enrichment score, the gene expression matrix of the discovery cohort and gene sets derived from the KEGG pathway were used as input for gene set enrichment analysis (GSEA). The analysis was performed using the default parameters in R package GSEA_R (version 1.2) and adjusted p-values less than 0.25 were considered to be significant. An unsupervised method called Gene Set Variation Analysis (GSVA) by the R package GSVA (version 3.18) was also performed on the discovery cohort and gene sets derived from the KEGG pathway to estimate the variation of KEGG gene sets enrichment across the samples.

### CIBERSORT

The immune cell proportions in the sepsis datasets were estimated and if there were different immune cell proportions between the low- and high-score cohorts generated from the Drp1-PPI GS was evaluated. CIBERSORT (version 1.04) was used to assess the proportions of immune cell populations in the discovery dataset.

### Statistical Analysis

The R packages, ade4 (Version: 1.7-22) and pROC (Version: 1.18.5) were used to generate the PCA plots and the ROC curves. Values with false discovery rates (FDR) less than 0.05 were considered to be significant.

### Example 1—Generation of DEG-Based and PPI-Based Drp1-Associated Gene Signatures

First, the present invention seeks to identify genes related to Drp1 protein expression. To accomplish this, the publicly available GEO dataset containing the gene expression data from human cells where Drp1 was overexpressed and from control cells (GSE182710) were used. With this, 438 genes differentially expressed (DEGs) after Drp1 overexpression were identified, henceforth referred to as the DEG-based Drp1-associated genes (Table 1).

TABLE 1
List of all DEG-based Drp1-associated genes.
This table contains all the genes identified as
differentially expressed with Drp1 upregulation.

| Symbol | log2(fold change) |
|---|---|
| ARSD | 9.473 |
| ADARB2 | 8.615 |
| DNAJC15 | 7.446 |
| LOC105376634 | 7.123 |
| ZNF577 | 6.52 |
| MICU3 | 6.461 |
| SLC16A9 | 6.368 |
| ARSD-AS1 | 6.29 |
| LRRC2 | 5.615 |
| SMO | 5.509 |
| MGAT4C | 5.34 |
| CNTN1 | 5.235 |
| EMB | 4.568 |
| AFP | 4.509 |
| Clorf115 | 4.267 |
| RPE65 | 4.189 |
| MACROH2A2 | 3.976 |
| ZNF649 | 3.852 |
| HTRA1 | 3.825 |
| DMKN | 3.809 |
| OSR1 | 3.793 |
| PCSK1N | 3.691 |
| WT1 | 3.581 |
| PACERR | 3.57 |
| GJA1 | 3.552 |
| IGFBP5 | 3.52 |
| DKK2 | 3.445 |
| PLA2G4A | 3.415 |
| CCDC8 | 3.409 |
| PYGO1 | 3.389 |
| KRTAP2-4 | 3.372 |
| CACNA2D1 | 3.356 |
| SPP1 | 3.294 |
| TERT | 3.265 |
| ZFP69 | 3.125 |
| PARP8 | 3.106 |
| FABP5 | 3.03 |
| SLCO2A1 | 3.029 |
| NR4A3 | 3.024 |
| CASC15 | 2.914 |
| RGS2 | 2.905 |
| MARCHF4 | 2.839 |
| KRTAP2-2 | 2.827 |
| TCIM | 2.809 |
| VLDLR | 2.809 |
| NKD1 | 2.807 |
| GPR63 | 2.803 |
| PDE4A | 2.791 |
| PRKD1 | 2.744 |
| CELF2 | 2.742 |
| TMEM52B | 2.72 |
| CXCR4 | 2.694 |
| EDN1 | 2.66 |
| IL6 | 2.657 |
| GK | 2.648 |
| TEX56P | 2.634 |
| KRTAP2-3 | 2.631 |
| SLC16A10 | 2.596 |
| LSAMP | 2.561 |
| ACSS1 | 2.558 |
| ENOX1 | 2.537 |
| RNF125 | 2.523 |
| C5orf63 | 2.454 |
| KANK4 | 2.44 |
| KRTAP2-1 | 2.426 |
| PYCARD-AS1 | 2.41 |
| FGFR2 | 2.401 |
| CXCL2 | 2.398 |
| PYCARD | 2.378 |
| ECI2 | 2.377 |
| USP44 | 2.373 |
| PLXNA4 | 2.314 |
| DIRAS1 | 2.307 |
| LOC105371332 | 2.295 |
| LINC01629 | 2.285 |
| SLC7A2 | 2.268 |
| FBN2 | 2.25 |
| HOTS | 2.233 |
| SACS | 2.232 |
| CR2 | 2.226 |
| TENM4 | 2.224 |
| SOSTDC1 | 2.218 |
| POPDC3 | 2.207 |
| LOC112267876 | 2.18 |
| FAT3 | 2.171 |
| NGF | 2.17 |
| KCNF1 | 2.165 |
| CXCL8 | 2.145 |
| SLC6A17 | 2.138 |
| MARK1 | 2.125 |
| CALHM6 | 2.121 |
| PAQR9 | 2.114 |
| ANKRD18DP | 2.091 |
| FRG1CP | 2.09 |
| MIR675 | 2.087 |
| KCNJ12 | 2.087 |
| NREP | 2.079 |
| DTNA | 2.056 |
| SLC2A3 | 2.038 |
| FBXO2 | 2.022 |
| ERVMER34-1 | 2.009 |
| RFLNB | 2.008 |
| OLR1 | 2.007 |
| LOC102724852 | 1.975 |
| H19 | 1.953 |
| NEURL1 | 1.948 |
| CXCL3 | 1.94 |
| STX11 | 1.926 |
| NELL2 | 1.925 |
| GIPR | 1.916 |
| KRT6C | 1.907 |
| MYH15 | 1.899 |
| EFR3B | 1.892 |
| SNORA65 | 1.862 |
| RASSF10 | 1.855 |
| FOXD1 | 1.843 |
| RBM11 | 1.841 |
| SLITRK5 | 1.831 |
| NPR3 | 1.823 |
| TRIM9 | 1.818 |
| JPH1 | 1.811 |
| ZNF182 | 1.798 |
| KLHL13 | 1.794 |
| HES7 | 1.79 |
| LOC107985236 | 1.774 |
| LOC101928004 | 1.773 |
| TNFSF18 | 1.746 |
| AREG | 1.742 |
| LRP3 | 1.735 |
| UPP1 | 1.727 |
| NEK3 | 1.727 |
| POLR3G | 1.721 |
| FAM184A | 1.719 |
| LRFN1 | 1.709 |
| COCH | 1.702 |
| EREG | 1.701 |
| KLHL4 | 1.696 |
| SPHK1 | 1.688 |
| KLRG2 | 1.677 |
| NUAK2 | 1.674 |
| LINC01291 | 1.673 |
| PTHLH | 1.667 |
| OXCT2 | 1.658 |
| GEM | 1.653 |
| SNORA69 | 1.65 |
| LOC102724428 | 1.648 |
| PTGS2 | 1.647 |
| KRT6A | 1.647 |
| NPTX1 | 1.646 |
| TLL2 | 1.641 |
| MIR132 | 1.639 |
| ZBTB8B | 1.638 |
| SCML2 | 1.636 |
| SIK1 | 1.632 |
| IGFBP2 | 1.63 |
| SERPINE1 | 1.623 |
| LARGE2 | 1.622 |
| GLI2 | 1.618 |
| TFAM | 1.616 |
| TNFAIP3 | 1.604 |
| C9orf47 | 1.604 |
| SLC5A4-AS1 | 1.601 |
| WNT10B | 1.594 |
| TRAM1L1 | 1.585 |
| CCN2 | 1.577 |
| MIR100HG | 1.577 |
| SLIT2 | 1.576 |
| LRRC8C | 1.572 |
| TMED6 | 1.571 |
| SNORA66 | 1.568 |
| LONRF3 | 1.563 |
| SLIT3 | 1.551 |
| LRP4-AS1 | 1.541 |
| C1orf21 | 1.536 |
| KLF6 | 1.527 |
| PALD1 | 1.526 |
| ALMS1-IT1 | 1.525 |
| SLAIN1 | 1.523 |
| LOC101929470 | 1.521 |
| NDRG2 | 1.513 |
| BHLHE40-AS1 | 1.509 |
| P2RY1 | 1.505 |
| PADI2 | 1.496 |
| SAP30 | 1.483 |
| LOC112268114 | 1.48 |
| LOC107986872 | 1.479 |
| SPRR1B | 1.474 |
| CCDC40 | 1.474 |
| LOC105376364 | 1.472 |
| FOXP4-AS1 | 1.467 |
| ANGPTL4 | 1.465 |
| RND1 | 1.462 |
| LOC112268270 | 1.462 |
| ITPR1 | 1.461 |
| TMEM158 | 1.461 |
| AKAP12 | 1.46 |
| FBXO6 | 1.454 |
| PAICS | 1.453 |
| GPC3 | 1.45 |
| IGFBP7-AS1 | 1.446 |
| CKB | 1.442 |
| TMCC2 | 1.441 |
| SOX21-AS1 | 1.438 |
| GNB4 | 1.432 |
| FOSL1 | 1.431 |
| NRCAM | 1.426 |
| INHBA | 1.423 |
| KIAA1755 | 1.423 |
| DEGS1 | 1.422 |
| AARD | 1.418 |
| PER2 | 1.416 |
| LOC107986196 | 1.412 |
| PCCA-DT | 1.409 |
| HSPA12A | 1.407 |
| SH2D5 | 1.404 |
| ZNF469 | 1.384 |
| SLC4A7 | 1.378 |
| LOC105370337 | 1.378 |
| RIMS2 | 1.375 |
| SNORD21 | 1.372 |
| PHF21B | 1.37 |
| FGF2 | 1.366 |
| LINC01293 | 1.362 |
| NRG1 | 1.362 |
| RBM20 | 1.362 |
| MARCHF1 | 1.359 |
| SLC16A14 | 1.357 |
| SOX21 | 1.356 |
| ITGB1-DT | 1.355 |
| DSCC1 | 1.353 |
| LOC112267943 | 1.352 |
| LOC105376475 | 1.352 |
| LOC102724808 | 1.351 |
| DOCK4 | 1.341 |
| LYAR | 1.339 |
| KCNQ5 | 1.334 |
| SNORA21B | 1.334 |
| CEP85L | 1.329 |
| LRP4 | 1.327 |
| DIRC3 | 1.324 |
| FOXG1 | 1.323 |
| TBC1D30 | 1.317 |
| AUTS2 | 1.314 |
| NOG | 1.314 |
| SNORA105A | 1.312 |
| FGF12 | 1.31 |
| LOC107985297 | 1.31 |
| PRXL2B | 1.307 |
| CREB5 | 1.305 |
| CRYZL2P | 1.302 |
| MIG7 | 1.3 |
| RAB29 | 1.298 |
| FAM27B | 1.298 |
| NAT8L | 1.297 |
| RBPMS2 | 1.294 |
| LOC105378954 | 1.293 |
| BMP7 | 1.293 |
| RAI14 | 1.288 |
| BHLHE40 | 1.285 |
| FGFBP1 | 1.284 |
| FOXF2 | 1.282 |
| S1PR3 | 1.282 |
| ETV5 | 1.28 |
| ZNF483 | 1.274 |
| GAREM2 | 1.273 |
| PPP1R3C | 1.271 |
| GAL | 1.269 |
| SLC25A19 | 1.266 |
| BARX2 | 1.265 |
| TRPC4 | 1.265 |
| NOVA1 | 1.265 |
| TWIST2 | 1.264 |
| PGF | 1.26 |
| NDUFAF4 | 1.257 |
| DUSP5 | 1.254 |
| LOC105371441 | 1.249 |
| MYB | 1.248 |
| ARHGEF26 | 1.247 |
| LOC105369349 | 1.242 |
| HAUS7 | 1.241 |
| WDR17 | 1.24 |
| MIR621 | 1.237 |
| LOC107984192 | 1.234 |
| BMP6 | 1.228 |
| LOC107984343 | 1.227 |
| SH3D19 | 1.226 |
| JAG1 | 1.22 |
| FREM1 | 1.214 |
| ABCC4 | 1.213 |
| CDC25A | 1.212 |
| NDUFAF8 | 1.212 |
| ANKRD20A2P | 1.211 |
| NOCT | 1.206 |
| FZD9 | 1.206 |
| GPR3 | 1.202 |
| KCNJ2 | 1.202 |
| LOC105375728 | 1.198 |
| BTNL9 | 1.197 |
| LOC105370935 | 1.197 |
| COL4A1 | 1.196 |
| LOC390877 | 1.195 |
| SNORD63 | 1.194 |
| PCDH9 | 1.191 |
| COL4A2-AS1 | 1.191 |
| ANKRD20A3P | 1.19 |
| LOC107984833 | 1.187 |
| PDCD2L | 1.185 |
| TRIP13 | 1.18 |
| LOC102724434 | 1.175 |
| ZNF738 | 1.174 |
| CHD5 | 1.173 |
| SRM | 1.171 |
| HES6 | 1.163 |
| NR4A1 | 1.162 |
| F11-AS1 | 1.16 |
| SMAD7 | 1.159 |
| EFNB2 | 1.157 |
| CHAC2 | 1.151 |
| SOWAHC | 1.151 |
| WEE1 | 1.149 |
| KRT6B | 1.148 |
| DDIAS | 1.147 |
| ANKRD20A1 | 1.143 |
| PALS2 | 1.14 |
| RFC3 | 1.14 |
| STEAP1B-AS1 | 1.139 |
| PDE4D | 1.137 |
| SLC16A12 | 1.132 |
| PLCB4 | 1.132 |
| PER1 | 1.129 |
| KIAA0930 | 1.129 |
| SGK1 | 1.126 |
| MYC | 1.125 |
| LINC00707 | 1.123 |
| ZIC5 | 1.123 |
| MTARC1 | 1.12 |
| SCARNA22 | 1.119 |
| DPF1 | 1.119 |
| VIM | 1.118 |
| TEX30 | 1.117 |
| ANKRD33B | 1.112 |
| ZFHX4 | 1.111 |
| URB2 | 1.109 |
| GBX2 | 1.106 |
| EXO1 | 1.104 |
| BRIX1 | 1.104 |
| FBXO45 | 1.102 |
| TFDP1 | 1.102 |
| CA2 | 1.101 |
| DUSP4 | 1.1 |
| STX2 | 1.1 |
| LHX1 | 1.099 |
| LOC105374838 | 1.095 |
| PTGER4 | 1.095 |
| CHDH | 1.094 |
| GNG11 | 1.092 |
| KREMEN2 | 1.092 |
| MIR658 | 1.092 |
| CCN1 | 1.091 |
| LOC101059954 | 1.091 |
| MN1 | 1.091 |
| LINC00941 | 1.09 |
| MTCL1 | 1.09 |
| DTL | 1.089 |
| LOC107984510 | 1.089 |
| NLN | 1.088 |
| SLC29A1 | 1.088 |
| SPRY4-AS1 | 1.087 |
| MIR1182 | 1.083 |
| CBX2 | 1.08 |
| PNO1 | 1.077 |
| LRFN4 | 1.077 |
| NUFIP1 | 1.076 |
| MAPK8IP1 | 1.071 |
| LOC112268106 | 1.07 |
| TMEM131L | 1.067 |
| EXO5-DT | 1.064 |
| AEN | 1.063 |
| LOC107985567 | 1.063 |
| ARLAC | 1.062 |
| SLC25A25 | 1.062 |
| PRPS1 | 1.062 |
| LOC105376380 | 1.061 |
| COL4A6 | 1.061 |
| PAK1IP1 | 1.06 |
| SLC30A3 | 1.059 |
| F2RL1 | 1.059 |
| LOC107985000 | 1.059 |
| SLC25A15 | 1.058 |
| CDCA7 | 1.057 |
| UTP15 | 1.057 |
| IL1A | 1.056 |
| CLDN14-AS1 | 1.056 |
| RASGEF1A | 1.055 |
| ANKRD1 | 1.054 |
| PRTG | 1.054 |
| RPP25 | 1.053 |
| KIAA1549L | 1.051 |
| MARS2 | 1.049 |
| LOC107984616 | 1.047 |
| PPP2R1B | 1.046 |
| BAMBI | 1.045 |
| CHPT1 | 1.043 |
| KBTBD6 | 1.041 |
| CUTC | 1.04 |
| TBC1D4 | 1.039 |
| PNPT1 | 1.038 |
| LUCAT1 | 1.037 |
| SLC29A4 | 1.037 |
| PITX2 | 1.036 |
| PARVB | 1.036 |
| PARVG | 1.036 |
| LEF1 | 1.035 |
| SNORD14C | 1.035 |
| CHST7 | 1.035 |
| LOC105369575 | 1.034 |
| IPO5 | 1.031 |
| HDGFL3 | 1.031 |
| TTC6 | 1.026 |
| MCTP2 | 1.026 |
| WNT5B | 1.024 |
| JADE1 | 1.021 |
| MCM10 | 1.021 |
| LINC02454 | 1.02 |
| FSD2 | 1.02 |
| LOC105378513 | 1.019 |
| ZNF695 | 1.018 |
| NUP160 | 1.016 |
| SDHB | 1.012 |
| ZNF618 | 1.012 |
| ATF3 | 1.011 |
| ANKRD13B | 1.009 |
| FAM86JP | 1.008 |
| TEX15 | 1.007 |
| NCS1 | 1.007 |
| LOC112268109 | 1.007 |
| POGLUT2 | 1.004 |
| BBS5 | 1.001 |

Next, a protein-protein interaction (PPI) analysis of the protein, Drp1, was performed to identify proteins predicted to interact with Drp1 using the STRING database. This method resulted in an additional 244 genes associated with Drp1, designated as PPI-based Drp1-associated genes (Table 2).

TABLE 2
List of all PPI-based Drp1-associated genes generated
from Drp1-DEG the STRING database.
This table contains all the genes identified as associated
with Drp1 using the STRING database.
PPI-based Drp1-associated genes

ABCD3
ACO2
ACTB
AFG3L2
AKAP1
AKT1
ALB
AMBRA1
AMPH
ANXA6
APAF1
APP
ATAD1
ATAD3A
ATG12
ATG14
ATG5
ATG7
ATL1
ATL2
ATL3
AVL9
BAX
BCAP31
BCL2
BCL2L1
BCL2L13
BECN1
BIK
BIN1
BNIP3
BNIP3L
CALCOCO2
CALM3
CALML3
CALML5
CAMK1
CANX
CASP1
CASP3
CASP8
CASP9
CD300C
CD47
CDK1
CDK5
CHCHD3
CLDN15
CLTC
CLTCL1
COX4I1
CS
CYCS
DENND10
DENND11
DENND6A
DENR
DIABLO
DIABLO-2
DNAJA3
DNAJC6
DNM1L
EIF2AK3
EPS15
ESRRA
FIS1
FLNA
FOXO3
FUNDC1
GABARAP
GABARAPL1
GABARAPL2
GAK
GAPDH
GDAP1
GDAP1L1
GSK3B
HADHB
HGS
HIF1A
HK2
HSP90AA1
HSP90AB1
HSPA1L
HSPA4
HSPA5
HSPA9
HSPD1
HTRA2
HTT
IMMT
INF2
INS
ITPR1
ITPR3
ITSN1
ITSN2
KIF5B
LONP1
LRRK2
MAOA
MAOB
MAP1A
MAP1B
MAP1LC3A
MAP1LC3B
MAPK1
MAPK3
MAPT
MARCHF5
MAVS
MBOAT2
MCU
MFF
MFN1
MFN2
MIEF1
MIEF2
MITF
MLKL
MT-CO1
MTFP1
MTOR
MUL1
NBR1
NFE2L2
NLRP3
NLRX1
NME4
NRF1
NSF
NUDT21
OMA1
OPA1
OPA3
OPTN
PACRG
PACS2
PARK7
PARL
PARL-2
PDHX
PEX11A
PEX11B
PEX11G
PEX13
PEX14
PEX16
PEX19
PEX3
PEX5
PEX6
PGAM5
PHB
PHB2
PIK3C3
PINK1
PKM
PKP3
PLD6
PLEK
PLEK2
POLG
PPARG
PPARGC1A
PPARGC1B
PPIF
PPP2CA
PRDX3
PRKACA
PRKACB
PRKACG
PRKN
PRNP
PSEN1
PSMD14
PTEN
PXDN
PXDNL
RAB32
RAB6A
RHOT1
RHOT2
RIPK1
RIPK3
RMDN3
ROCK1
SACS
SAMM50
SDHA
SENP3
SENP5
SH3GL1
SH3GL2
SH3GLB1
SH3GLB2
SIRPA
SIRT1
SIRT3
SLA
SLC25A46
SLC25A5
SNCA
SNPH
SOD1
SOD2
SOD2-2
SPG7
SQSTM1
STX17
STXBP1
SUMO1
SUMO2
SYNCRIP
SYNJ1
TARDBP
TBC1D15
TFAM
TFEB
TIMM23
TOMM20
TOMM22
TOMM40
TOMM70
TP53
TRAK1
TRAK2
TXN
UBE2E1
UBE2E3
UCP2
ULK1
UQCRC2
USP14
USP15
USP30
VAPB
VCP
VDAC1
VDAC2
VPS26A
VPS35
VPS45
YME1L1

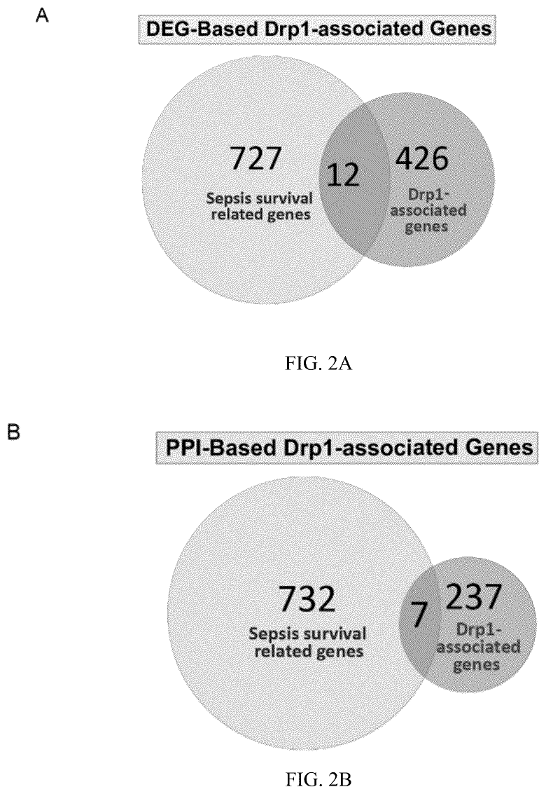

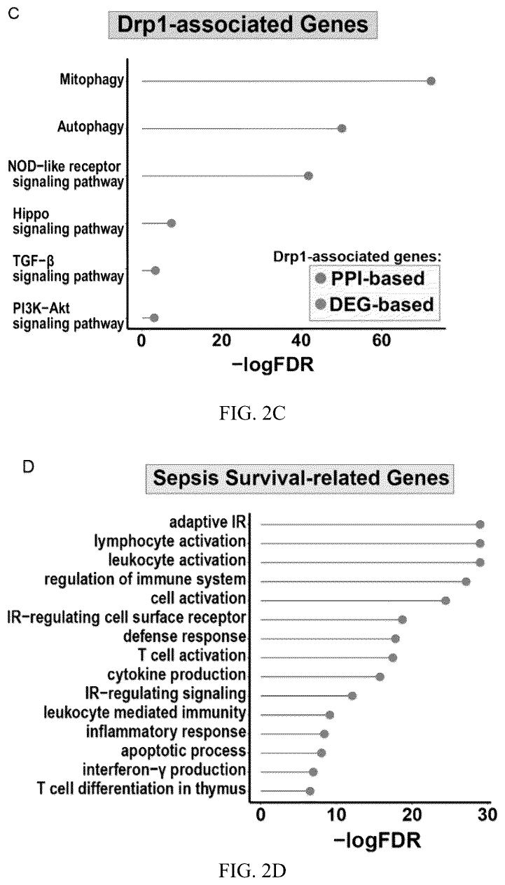

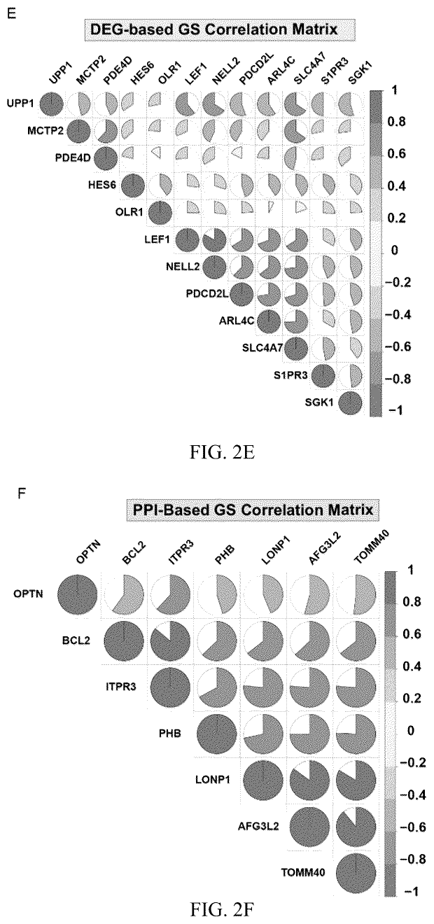

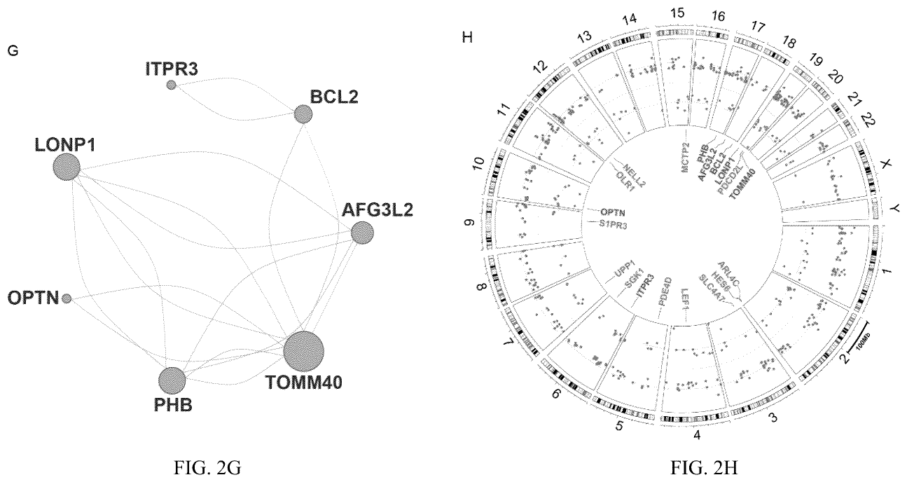

These two sets of Drp1-associated genes were then enriched with genes related to sepsis survival. This revealed 12 and 7 common genes between the sepsis survival genes and the DEG- or PPI-based Drp1-associated gene sets, respectively (FIGS. 2A-2B, Table 3). The 12 and 7 gene sets were designated as DEGs and PPI Drp1-gene signatures (GS), respectively.

TABLE 3
List of genes in the Drp1-associated gene signatures.
Two gene signatures were generated: Drp1-DGEs GS and Drp1-PPI GS.
Drp1-associated Genes

| GS Name | Gene | |
|---|---|---|
| DEG- | SLC4A7 | Solute Carrier Family 4 Member 7 |
| based | PDCD2L | Programmed Cell Death 2-Like |
| GS | UPP1 | Uridine Phosphorylase 1 |
| | ARLAC | ADP-Ribosylation Factor-Like 4C |
| | NELL2 | Neural EGFL-Like 2 |
| | LEF1 | Lymphoid Enhancer-Binding Factor 1 |
| | SGK1 | Serum/Glucocorticoid-Regulated Kinase 1 |
| | PDE4D | Phosphodiesterase 4D |
| | S1PR3 | Sphingosine-1-Phosphate Receptor 3 |
| | MCTP2 | Multiple C2 Domains Transmembrane Protein 2 |
| | HES6 | Hes Family BHLH Transcription Factor 6 |
| | OLR1 | Oxidized Low-Density Lipoprotein Receptor 1 |
| PPI- | AFG3L2 | AFG3-Like Matrix AAA Peptidase Subunit 2 |
| based | BCL2 | B-Cell Lymphoma 2 |
| GS | ITPR3 | Inositol 1,4,5-Trisphosphate Receptor Type 3 |
| | LONP1 | Lon Peptidase 1, Mitochondrial |
| | OPTN | Optineurin |
| | PHB | Prohibitin |
| | TOMM40 | Translocase of Outer Mitochondrial Membrane 40 |

### Example 2—Kyoto Encyclopedia of Genes and Genomes (KEGG) Analysis

KEGG pathway analysis was performed using both the Drp1 DEGs and PPI gene signatures (FIG. 2C). This revealed 6 pathways that were enriched in these genes: mitophagy, autophagy, the NOD-like receptor, TGF-β, and PI3K-Akt signaling pathways.

A gene signature that differentiates between patients with a low or high risk of sepsis-related death has been previously published and a list of genes related to sepsis survival has been compiled (Feng, A. et al., NAMPT-associated gene signature in the prediction of severe sepsis. Am J Transl Res 2022, 14, (10), 7090-7097). KEGG analysis of these genes revealed 15 pathways enriched in sepsis-survival genes, consisting of primarily immunomodulatory pathways (FIG. 2D).

### Example 3—Validation of Generated PPI-Based Drp1-Associated Gene Signature

The correlation of the Drp1-associated genes with the other genes in their respective GS were assessed to validate that PPI GS was generated correctly (FIGS. 2E-2F). As expected, the Drp1-DEGs GS consisted of genes with relatively low correlation with one another (FIG. 2E). On the other hand, the Drp1-PPI GS consisted of genes that are highly correlated with one another (FIG. 2F).

The PPI network of this GS was shown in FIG. 2G, meanwhile, no connection was found within Drp1-DEGs GS. The location of the genes of both GS were shown in FIG. 2H, with the Drp1-DEGs GS represented by blue lettering, and the Drp1-PPI GS represented by red lettering in the inner circle. The dots in the middle circle represented the locations of genes related to sepsis survival, with red dots representing upregulated and blue dots representing downregulated sepsis survival-related genes.

### Example 4—Evaluation of Prognostic Capabilities of GS

The present invention seeks to evaluate both GS for their capabilities of distinguishing high and low sepsis risk groups.

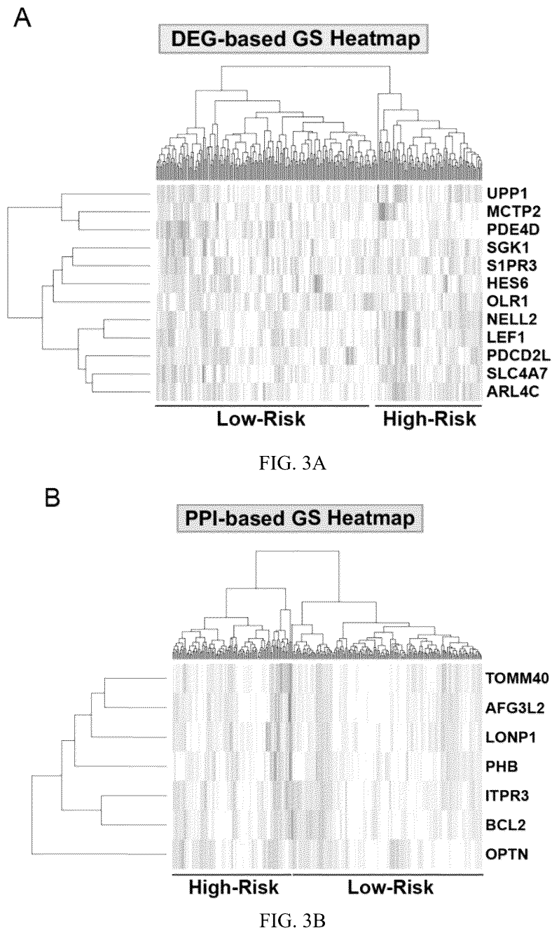

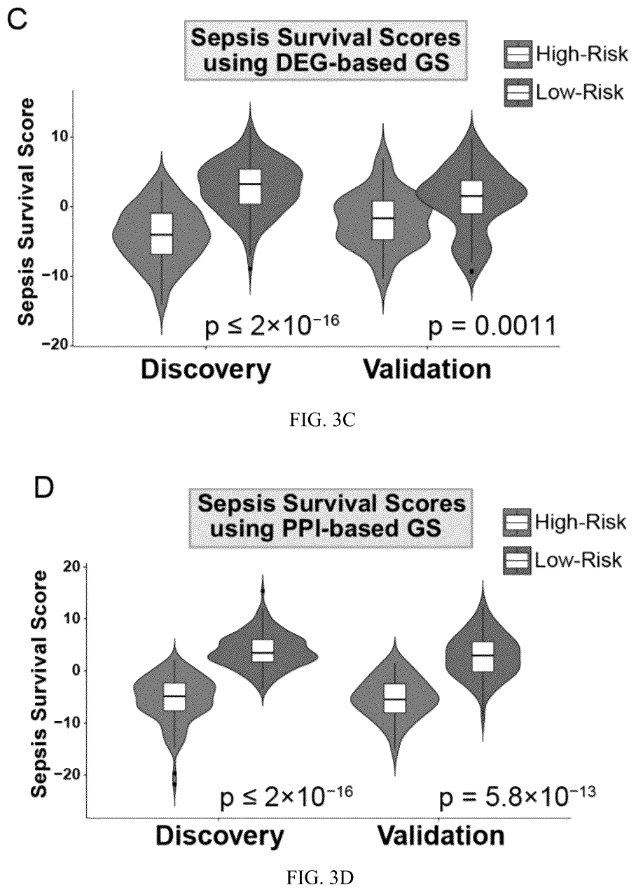

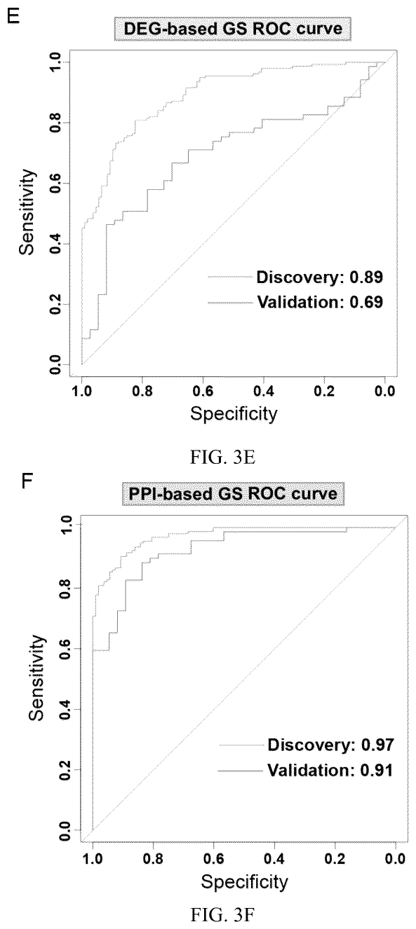

To accomplish this, the expression levels of the individual genes within the GS were first compiled and the low and high-risk patients were separated. Heatmaps of the DEGs-based (FIG. 3A) and PPI-based (FIG. 3B) GS were obtained, which both showed expression patterns that differentiated low and high-risk sepsis groups from the discovery cohort.

To confirm this differentiation, violin plots of the sepsis survival scores of both gene signatures in the discovery and validation cohorts were generated. Sepsis survival scores were calculated using the formula described above, which assessed the probability of septic death in each patient. Lower score values indicated a worse prognosis and survival rate, this group was labeled as the high-risk septic patients. Higher scores were classified as low-risk septic patients. Both sepsis survival score violin plots demonstrated that the DEG-based (FIG. 3C) and the PPI-based (FIG. 3D) GS successfully differentiated between low and high-risk sepsis patients, with the PPI-based GS providing greater separation between the low and high-risk groups.

To evaluate both the specificity and sensitivity of the gene signatures, a receiver operating characteristic curve (ROC) was generated for the DEG-based (FIG. 3E) and PPI-based (FIG. 3F) GS. These ROC curves revealed an AUC value of 0.89 for the discovery cohort using the DEG-based GS and 0.97 using the PPI-based GS. For the validation cohort, the DEG-based GS had an AUC value of 0.69 and the PPI-based GS had a value of 0.91, indicating that the PPI-based GS was more specific and sensitive compared to the DEG-based GS.

### Example 5—Principal Component Analysis

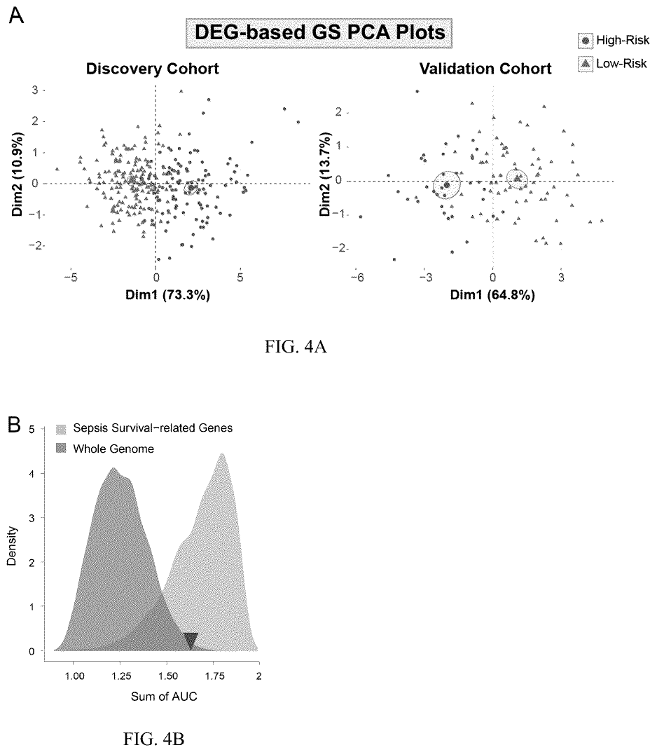

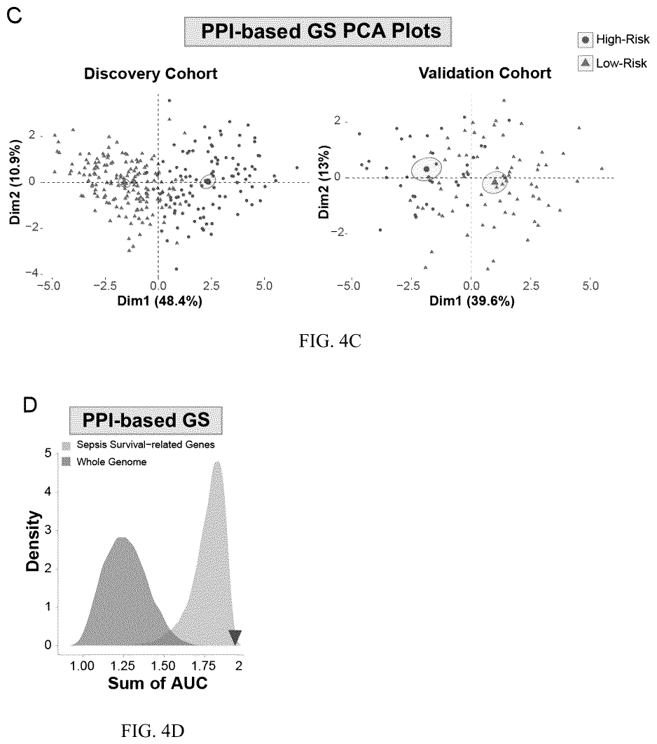

Principal component analysis (PCA) of both gene signatures showed that the DEG-based (FIG. 4A) and PPI-based (FIG. 4C) GS can completely differentiate high-risk sepsis patients from low-risk patients in both the discovery and validation cohorts. The PCA analysis for DEG-based GS represented 78.5-84.2% of the variable expression data. The PCA analysis for PPI-based GS represented 52.6-59.3% of the variable expression data.

To confirm that both gene signatures are more predictive of sepsis risk than a random set of whole genome genes (blue) or a random set of sepsis-survival genes (yellow), the density distribution of the AUC was plotted for the whole genome and sepsis-survival genes (FIGS. 4B and 4D). The sum of the DEG-based GS AUC value (1.58) and the sum of the Drp1-PPI GS AUC value (1.88) were indicated by the red inverted triangles. These results indicated that both the DEG-based and PPI-based GS had a prognostic power greater than random genes from the whole genome. However, the PPI-based GS had prognostic power that was greater than that of other random genes related to sepsis survival, while the DEG-based GS did not. Therefore, the PPI-based GS was selected to calculate sepsis survival scores for the patients in the discovery cohort.

### Example 6—KEGG, GSVA, and GSEA Analysis

To understand the cellular and molecular mechanism of the prognostic power of the PPI-GS, the transcriptomic and cellularity differences between the high-score and low-score groups were compared. Using the PPI-based GS, the patients were then re-clustered into high-survival score (Table 4) or low-survival score (Table 5) cohorts based on whether the survival scores of these samples were higher or lower than the medium survival score across all samples.

TABLE 4
High Gene Signature Score: Low-risk Cohort.
The patients in the discovery cohort were reorganized according to the risk
score generated using the PPI-based GS. High scores indicate a better
sepsis prognosis.

| Patient ID | Risk Score | State | |
|---|---|---|---|
| X6235804015_K | 15.32907863 | High-Score | Lowest Predicted |
| X6235821067_G | 11.9019953 | High-Score | Sepsis Risk |
| X6247207028_C | 11.89040393 | High-Score | |
| X6247207024_G | 11.19112324 | High-Score | |
| X6235821083_D | 10.50942706 | High-Score | |
| X6235821099_G | 10.36732368 | High-Score | |
| X6247215003_G | 10.22918569 | High-Score | |
| X6235821067_I | 9.263411035 | High-Score | |
| X6235821067_J | 9.122500431 | High-Score | |
| X6235821066_E | 8.846564055 | High-Score | |
| X6235821066_K | 8.765043981 | High-Score | |
| X6235804015_H | 8.752206757 | High-Score | |
| X6247207028_K | 8.571888504 | High-Score | |
| X6247215001_J | 8.328851147 | High-Score | |
| X6247207026_C | 8.223416076 | High-Score | |
| X6247207100_I | 8.147900523 | High-Score | |
| X6247207027_B | 8.144834462 | High-Score | |
| X6235804002_B | 7.966103036 | High-Score | |
| X6247207022_K | 7.922870946 | High-Score | |
| X6247207026_D | 7.921827363 | High-Score | |
| X6247215003_E | 7.748357847 | High-Score | |
| X6247207028_L | 7.372622602 | High-Score | |
| X6235804002_I | 6.905334247 | High-Score | |
| X6235821091_F | 6.882104521 | High-Score | |
| X6235804014_K | 6.857036191 | High-Score | |
| X6235821084_E | 6.814345872 | High-Score | |
| X6235821084_A | 6.776576093 | High-Score | |
| X6247207027_L | 6.633154002 | High-Score | |
| X6235821091_A | 6.518393645 | High-Score | |
| X6235821084_B | 6.441678821 | High-Score | |
| X6235821099_J | 6.340289691 | High-Score | |
| X6247207062_B | 6.330958112 | High-Score | |
| X6247215003_A | 6.324379923 | High-Score | |
| X6235821101_L | 6.299316052 | High-Score | |
| X6247207066_K | 6.272154924 | High-Score | |
| X6247215003_I | 6.246146177 | High-Score | |
| X6247207028_F | 6.215366341 | High-Score | |
| X6247207066_I | 6.205455736 | High-Score | |
| X6235821091_H | 6.156693019 | High-Score | |
| X6235821099_I | 6.031854773 | High-Score | |
| X6247207062_F | 5.910123841 | High-Score | |
| X6235821084_F | 5.881792061 | High-Score | |
| X6247215001_G | 5.762696648 | High-Score | |
| X6235804014_J | 5.731933813 | High-Score | |
| X6247207064_J | 5.697833915 | High-Score | |
| X6235804002_D | 5.697140152 | High-Score | |
| X6247207024_C | 5.670663385 | High-Score | |
| X6235804004_F | 5.553937976 | High-Score | |
| X6235821101_A | 5.542310426 | High-Score | |
| X6235821066_L | 5.521646637 | High-Score | |
| X6235821079_K | 5.443884148 | High-Score | |
| X6247207030_D | 5.398765802 | High-Score | |
| X6247207066_D | 5.342974193 | High-Score | |
| X6235804017_L | 5.299670924 | High-Score | |
| X6247207032_K | 5.276401144 | High-Score | |
| X6235804014_H | 5.229396908 | High-Score | |
| X6247215001_H | 5.053688934 | High-Score | |
| X6235804004_B | 4.967954123 | High-Score | |
| X6247207022_I | 4.937467294 | High-Score | |
| X6247207043_D | 4.724892715 | High-Score | |
| X6247207062_G | 4.659694368 | High-Score | |
| X6235821066_D | 4.638556103 | High-Score | |
| X6247207022_J | 4.618264858 | High-Score | |
| X6235821101_E | 4.424784262 | High-Score | |
| X6247207025_B | 4.368474959 | High-Score | |
| X6247207025_A | 4.309864336 | High-Score | |
| X6235821079_G | 3.976760144 | High-Score | |
| X6247215003_F | 3.971779535 | High-Score | |
| X6247207028_H | 3.954178442 | High-Score | |
| X6235821095_H | 3.805230288 | High-Score | |
| X6235821080_D | 3.797767295 | High-Score | |
| X6247207043_A | 3.636928436 | High-Score | |
| X6247207043_L | 3.611370105 | High-Score | |
| X6235821066_A | 3.582325189 | High-Score | |
| X6247207030_G | 3.525915756 | High-Score | |
| X6247207025_F | 3.498634276 | High-Score | |
| X6247207064_K | 3.471608202 | High-Score | |
| X6247207028_E | 3.461854598 | High-Score | |
| X6247207066_G | 3.429247323 | High-Score | |
| X6235821084_J | 3.413499778 | High-Score | |
| X6235821102_F | 3.369732609 | High-Score | |
| X6235821067_C | 3.348049144 | High-Score | |
| X6247215001_A | 3.279879298 | High-Score | |
| X6247215001_F | 3.224123944 | High-Score | |
| X6235804015_C | 3.080607951 | High-Score | |
| X6247207025_G | 3.068852558 | High-Score | |
| X6235804015_I | 3.06820566 | High-Score | |
| X6235821099_D | 3.063426177 | High-Score | |
| X6247207066_C | 3.021075964 | High-Score | |
| X6247207032_B | 2.997618365 | High-Score | |
| X6235821102_B | 2.978000738 | High-Score | |
| X6235821079_B | 2.889327959 | High-Score | |
| X6235821090_J | 2.842975122 | High-Score | |
| X6247207022_C | 2.725262484 | High-Score | |
| X6235821066_C | 2.722141525 | High-Score | |
| X6247207066_L | 2.711228012 | High-Score | |
| X6247207024_K | 2.640066572 | High-Score | |
| X6247207064_D | 2.471479795 | High-Score | |
| X6247207100_K | 2.400092956 | High-Score | |
| X6235821084_H | 2.356286996 | High-Score | |
| X6235821090_D | 2.307125989 | High-Score | |
| X6235821095_L | 2.299192989 | High-Score | |
| X6247207062_J | 2.271993703 | High-Score | |
| X6247207032_J | 2.263715884 | High-Score | |
| X6247207062_E | 2.263009782 | High-Score | |
| X6247207066_A | 2.258183517 | High-Score | |
| X6247207064_F | 2.161938968 | High-Score | |
| X6247207030_C | 2.142478396 | High-Score | |
| X6247207030_A | 2.102804919 | High-Score | |
| X6235821095_E | 2.067036322 | High-Score | |
| X6235821102_L | 2.043173999 | High-Score | |
| X6235804014_E | 1.987959736 | High-Score | |
| X6247207022_E | 1.92573676 | High-Score | |
| X6235821091_K | 1.845484971 | High-Score | |
| X6235821090_C | 1.843623376 | High-Score | |
| X6235821101_H | 1.816030459 | High-Score | |
| X6247207027_J | 1.796665795 | High-Score | |
| X6235821101_I | 1.706578568 | High-Score | |
| X6235804004_A | 1.683448096 | High-Score | |
| X6235821101_B | 1.63437136 | High-Score | |
| X6247207100_D | 1.616810058 | High-Score | |
| X6235804002_H | 1.453705168 | High-Score | |
| X6247207028_J | 1.431075287 | High-Score | |
| X6235821080_E | 1.395684227 | High-Score | |
| X6235821067_B | 1.38534751 | High-Score | |
| X6247207024_B | 1.380190467 | High-Score | |
| X6247215001_C | 1.336826581 | High-Score | |
| X6235821095_D | 1.170663798 | High-Score | |
| X6235821099_C | 1.166927782 | High-Score | |
| X6235821102_A | 1.090952863 | High-Score | |
| X6247207064_A | 1.032825163 | High-Score | |
| X6247207030_L | 0.964783156 | High-Score | |

TABLE 5
Low Gene Signature Score: High-risk Cohort.
List of patients from the discovery cohort with low-risk scores generated
from the PPI-based GS. Low scores indicate a worse sepsis prognosis.

| Patient ID | Risk Score | State | |
|---|---|---|---|
| X6235804017_A | −21.80022592 | Low-Score | Highest Predicted |
| X6235821091_L | −19.6844397 | Low-Score | Sepsis Risk |
| X6247207025_L | −14.75745237 | Low-Score | |
| X6247207022_G | −14.34351213 | Low-Score | |
| X6235821079_F | −14.12778855 | Low-Score | |
| X6247207043_B | −13.89161151 | Low-Score | |
| X6247207022_A | −13.72446959 | Low-Score | |
| X6235821083_K | −12.49873491 | Low-Score | |
| X6235821084_L | −12.30926369 | Low-Score | |
| X6235821080_A | −12.13320052 | Low-Score | |
| X6235804014_L | −11.80636745 | Low-Score | |
| X6235804015_E | −11.76268087 | Low-Score | |
| X6247207027_A | −11.26035413 | Low-Score | |
| X6235821066_H | −10.97338102 | Low-Score | |
| X6235821080_I | −10.95412186 | Low-Score | |
| X6247215003_K | −10.11554571 | Low-Score | |
| X6247207100_C | −9.718277013 | Low-Score | |
| X6247207043_I | −9.547035296 | Low-Score | |
| X6235804017_K | −9.282980593 | Low-Score | |
| X6235821083_F | −8.385371135 | Low-Score | |
| X6235821080_K | −8.320424296 | Low-Score | |
| X6247207064_C | −8.268175016 | Low-Score | |
| X6235821102_J | −8.205068028 | Low-Score | |
| X6247207062_L | −8.171810099 | Low-Score | |
| X6247207032_C | −7.831083761 | Low-Score | |
| X6235821091_I | −7.776189711 | Low-Score | |
| X6235804014_C | −7.740912545 | Low-Score | |
| X6247207027_K | −7.673694978 | Low-Score | |
| X6235821079_J | −7.574160598 | Low-Score | |
| X6235821099_B | −7.420837427 | Low-Score | |
| X6235804002_C | −7.318137219 | Low-Score | |
| X6247215001_L | −7.306514274 | Low-Score | |
| X6247207026_H | −7.280713148 | Low-Score | |
| X6235821084_I | −7.277588906 | Low-Score | |
| X6247207024_F | −7.129914018 | Low-Score | |
| X6247207100_L | −6.975280864 | Low-Score | |
| X6235804004_E | −6.969726239 | Low-Score | |
| X6247207032_H | −6.944175509 | Low-Score | |
| X6235821080_C | −6.888893559 | Low-Score | |
| X6235804017_I | −6.744422745 | Low-Score | |
| X6235821099_A | −6.594888064 | Low-Score | |
| X6247207030_E | −6.489221048 | Low-Score | |
| X6235804015_F | −6.439048184 | Low-Score | |
| X6247207032_D | −6.328008276 | Low-Score | |
| X6247207025_I | −6.158999948 | Low-Score | |
| X6247207064_B | −5.893327614 | Low-Score | |
| X6235804017_D | −5.868681821 | Low-Score | |
| X6247207043_J | −5.812205254 | Low-Score | |
| X6247207027_D | −5.739703772 | Low-Score | |
| X6235821099_K | −5.369944359 | Low-Score | |
| X6235821095_K | −5.339265097 | Low-Score | |
| X6235821079_A | −5.06509599 | Low-Score | |
| X6235804002_L | −4.969459199 | Low-Score | |
| X6247207028_G | −4.965337221 | Low-Score | |
| X6235821090_K | −4.868716338 | Low-Score | |
| X6235821102_D | −4.806340952 | Low-Score | |
| X6247207066_H | −4.745632038 | Low-Score | |
| X6235804014_A | −4.702592199 | Low-Score | |
| X6235804004_D | −4.512310986 | Low-Score | |
| X6235804004_J | −4.456689895 | Low-Score | |
| X6235821084_G | −4.375094622 | Low-Score | |
| X6247207026_I | −3.832682217 | Low-Score | |
| X6247207022_L | −3.74306703 | Low-Score | |
| X6247215003_C | −3.647749887 | Low-Score | |
| X6235821091_B | −3.561503936 | Low-Score | |
| X6247207062_I | −3.55118388 | Low-Score | |
| X6235821083_B | −3.453978899 | Low-Score | |
| X6247207043_K | −3.451227912 | Low-Score | |
| X6235821090_A | −3.427569053 | Low-Score | |
| X6235821095_G | −3.42268054 | Low-Score | |
| X6235821066_I | −3.417352387 | Low-Score | |
| X6235804014_F | −3.323683483 | Low-Score | |
| X6235821102_G | −3.306092479 | Low-Score | |
| X6235821080_H | −3.299377491 | Low-Score | |
| X6247207043_E | −3.235390751 | Low-Score | |
| X6235804002_F | −3.222549137 | Low-Score | |
| X6235821102_K | −2.998994894 | Low-Score | |
| X6235821083_C | −2.975010728 | Low-Score | |
| X6235804004_G | −2.77383765 | Low-Score | |
| X6247207066_F | −2.635263567 | Low-Score | |
| X6247207064_G | −2.510885695 | Low-Score | |
| X6247207100_E | −2.45746004 | Low-Score | |
| X6235821102_I | −2.447213101 | Low-Score | |
| X6247207028_A | −2.400548773 | Low-Score | |
| X6247207028_I | −2.37459805 | Low-Score | |
| X6247207100_H | −2.359742555 | Low-Score | |
| X6247207026_B | −2.132199143 | Low-Score | |
| X6235821080_J | −2.076363956 | Low-Score | |
| X6235821066_F | −2.067248881 | Low-Score | |
| X6247207100_B | −2.050408625 | Low-Score | |
| X6235821066_J | −2.020595724 | Low-Score | |
| X6247207026_J | −1.98181026 | Low-Score | |
| X6247207025_J | −1.891821678 | Low-Score | |
| X6247207024_H | −1.804358852 | Low-Score | |
| X6235804014_G | −1.801783855 | Low-Score | |
| X6235821083_H | −1.631962133 | Low-Score | |
| X6235821067_L | −1.608745047 | Low-Score | |
| X6247207022_F | −1.531030907 | Low-Score | |
| X6235821090_L | −1.307087984 | Low-Score | |
| X6235821079_L | −1.205146563 | Low-Score | |
| X6235804017_H | −1.203761808 | Low-Score | |
| X6247207030_I | −1.099594216 | Low-Score | |
| X6247207062_H | −1.084220107 | Low-Score | |
| X6247207030_B | −1.058971902 | Low-Score | |
| X6247207022_D | −0.964598382 | Low-Score | |
| X6247207024_D | −0.907905491 | Low-Score | |
| X6247207026_E | −0.709059605 | Low-Score | |
| X6235821090_G | −0.662197969 | Low-Score | |
| X6235821067_H | −0.542274531 | Low-Score | |
| X6235804017_J | −0.41167157 | Low-Score | |
| X6235821095_J | −0.408161736 | Low-Score | |
| X6235804002_E | −0.396631257 | Low-Score | |
| X6247207024_E | −0.362929869 | Low-Score | |
| X6247207027_H | −0.267266345 | Low-Score | |
| X6247215003_B | −0.220176172 | Low-Score | |
| X6235821084_D | −0.203077731 | Low-Score | |
| X6235821099_F | −0.151131631 | Low-Score | |
| X6247207066_J | −0.123652957 | Low-Score | |
| X6235821083_E | −0.024381196 | Low-Score | |
| X6235804015_D | 0.060143999 | Low-Score | |
| X6247215001_D | 0.110573219 | Low-Score | |
| X6235804014_B | 0.166681435 | Low-Score | |
| X6247207032_G | 0.206444852 | Low-Score | |
| X6235804017_G | 0.240393143 | Low-Score | |
| X6235804015_G | 0.261633298 | Low-Score | |
| X6235821067_D | 0.40251306 | Low-Score | |
| X6247207025_K | 0.448948878 | Low-Score | |
| X6235821090_F | 0.602465991 | Low-Score | |
| X6235821083_G | 0.697466675 | Low-Score | |
| X6247207027_E | 0.767627652 | Low-Score | |
| X6247207026_A | 0.770390941 | Low-Score | |
| X6235821101_F | 0.7778032 | Low-Score | |
| X6235821079_I | 0.949018472 | Low-Score | |

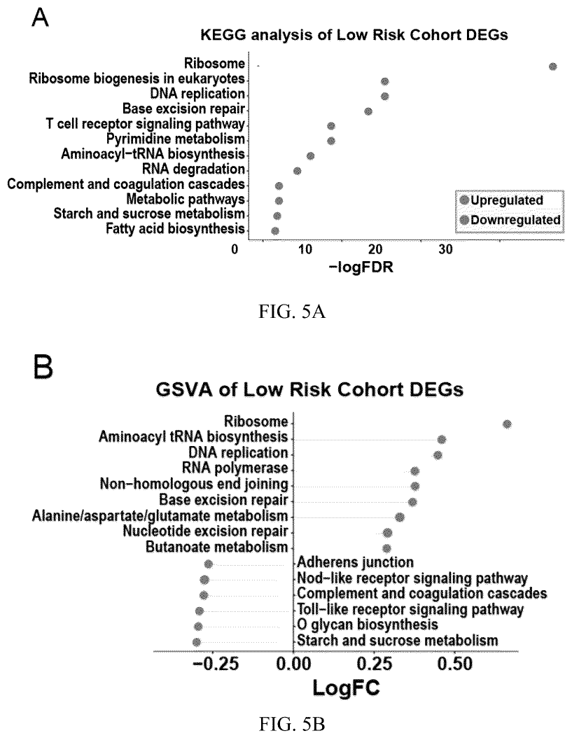

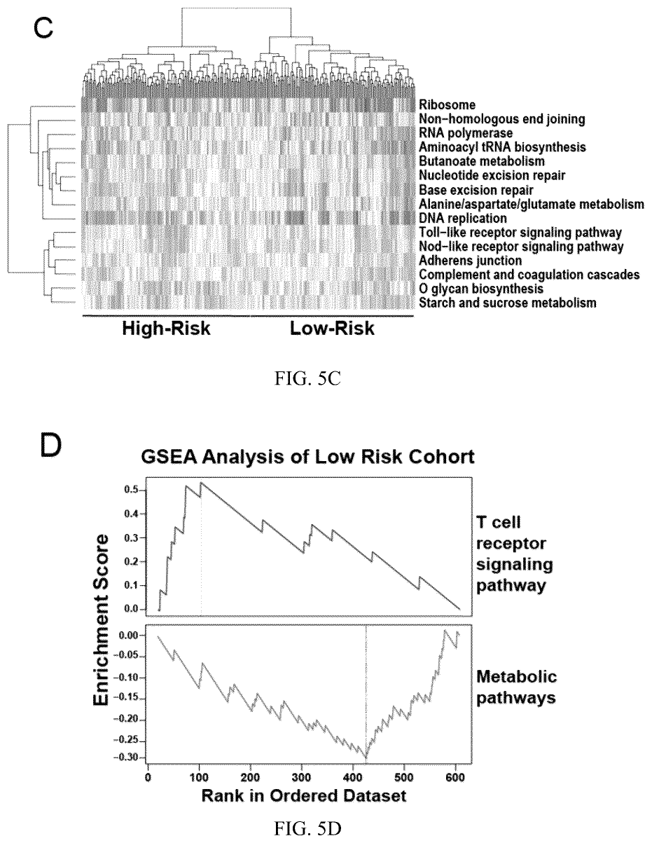

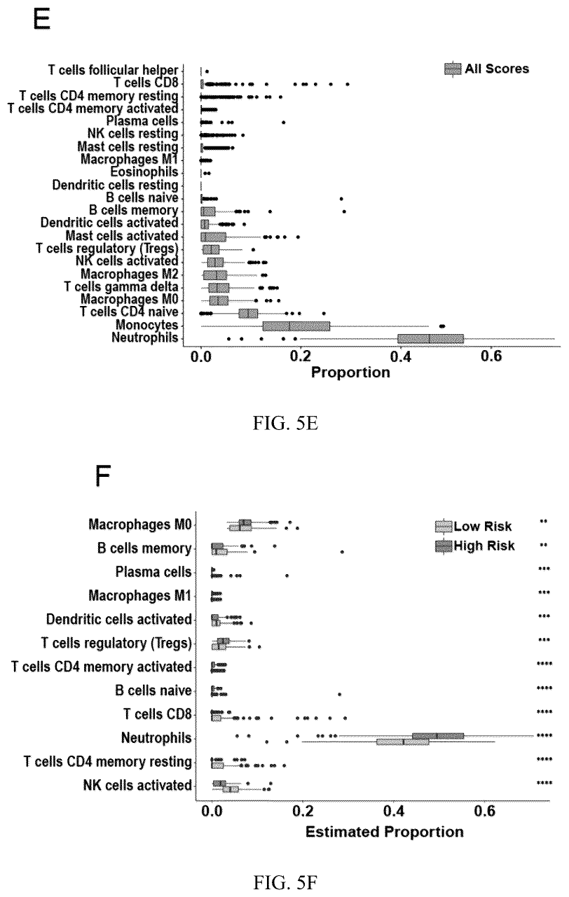

With the new cohorts generated from the PPI-based GS, DEGs in the high-survival score cohorts were identified in comparison to the low-survival score cohorts, where low-survival scores indicated a higher-risk group for sepsis severity. KEGG analysis showed that the upregulated genes in the high-score, low-risk cohort were enriched in the ribosome, DNA replication, T cell, and RNA pathways, while the downregulated genes in this lower-risk group were enriched in the metabolic and the complement and coagulation pathways (FIG. 5A).

Gene Set Variation Analysis (GSVA) was then utilized to identify additional pathways enriched in the high scores, lower-risk cohort (FIG. 5B). This was highly consistent with the KEGG analysis in DEGs. A heatmap of the pathways identified by GSVA was then generated. This revealed pathway enrichment patterns that could differentiate between low- and high-sepsis survival score patients (FIG. 5C).

Using gene set enrichment analysis (GSEA), it was found that the high-score, lower-risk cohort had increased T-cell signaling and decreased metabolic signaling (FIG. 5D).

Because the results from KEGG, GSVA, and GSEA all indicated an alteration in immune pathways (FIGS. 5A-5D), CIBERSORT was used to estimate the proportions of immune cells in the entire discovery cohort (FIG. 5E). The low- and high-risk cohorts were then separated from one another (FIG. 5F). This separation revealed a predicted immune profile shift between the low and high-risk groups. Specifically, the lower-risk patients were predicted to have decreased levels of neutrophils and increased activation of NK cells.

The present invention demonstrates that Drp1-associated proteins can predict sepsis severity, implicating mitochondrial dynamics in sepsis progression. Interestingly, the PPI-based GS predicted sepsis survival better than the DEG-based GS, as indicated by higher specificity and sensitivity values. A plausible explanation for this is that the PPI-based GS consists of proteins that interact with Drp1. Therefore, the present invention identifies genes that have more functional outcomes than genes identified by Drp1 overexpression. In the present invention, the STRING database was used to identify proteins that interact with Drp1, which generates functional protein association networks grouped into shells. In the analysis, only the primary protein interactors were used. The primary interactors represent proteins that are directly associated with Drp1. On the other hand, the secondary protein interactors (or the 2^{nd} shell) contain proteins that associate with primary protein interactors. In the present invention, mitochondrial dynamics are capable of predicting sepsis survival. The expression of proteins that are directly associated with Drp1 was evaluated.

In the present invention, datasets from two different biological sources were used: primary human peripheral blood mononuclear cells (PBMC) and human cell lines. Because the aim of the present invention was to evaluate the effects of altered mitochondrial dynamics on sepsis survival, the study was chosen to focus on the major fission regulator, Drp1, and proteins that are directly affected by Drp1.

To identify genes directly related to Drp1, human cell lines are necessary, as Drp1 deletion causes embryonic lethality in mice. Moreover, by using data from cells, only genes directly affected by Drp1 upregulation were gathering. Global upregulation of Drp1 in mice causes immense damage, which would convolute the analysis and cause such a high degree of dysfunction that the resulting data would no longer have biological applicability to sepsis. Existing PBMC expression data was then used to confirm that these genes are detectable and capable of differentiating between low- and high-risk patients. By using PBMC from septic patients, it was validated that the identified genes derived from the human cell lines are relevant in a clinical setting.

The present invention demonstrates that genes related to the major mitochondrial shaping protein, Drp1, can predict sepsis severity. For that reason, mitochondrial dynamics not only offers a prognostic opportunity but also provides a novel therapeutic strategy.

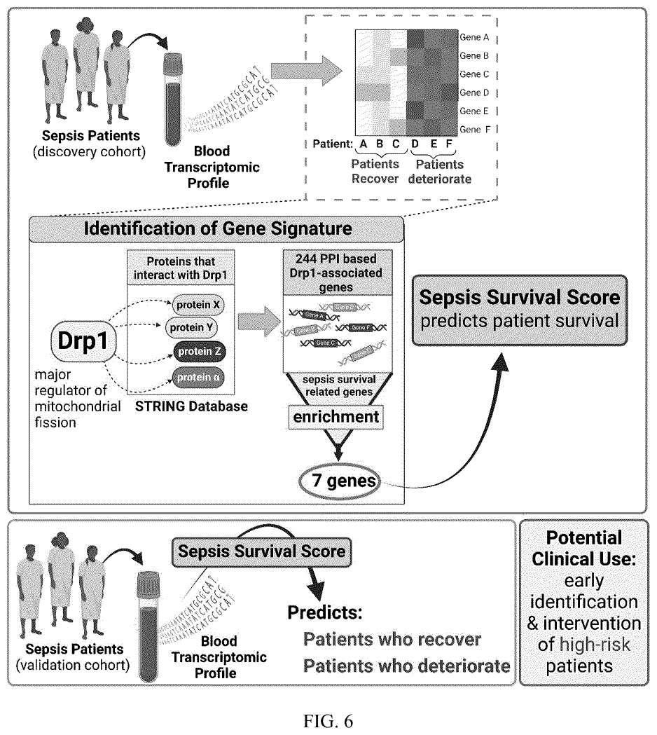

By engineering and validating gene signatures linked to Drp1, the present invention demonstrates that peripheral blood gene expression can accurately predict sepsis survival as well as recovery or deterioration for a patient, and thus enable early identification and intervention of high-risk patients (FIG. 6). This innovative sepsis prognostic biomarkers (Drp1-DEG GS and/or Drp1-PPI GS) can also be used to design treatment strategies.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. These examples should not be construed as limiting. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

1. A method of reducing sepsis severity in a subject, wherein the subject is human, comprising the following steps:
obtaining a sample from the subject, wherein the sample is blood;
detecting in the sample the expression levels of genes in dynamin-related protein 1-differentially expressed genes (Drp1-DEGs) gene signature and dynamin-related protein 1-protein-protein interaction (Drp1-PPI) gene signature;
calculating a first sepsis survival score as a linear combination of gene expression values and their corresponding weight values in the Drp1-DEGs gene signature and Drp1-PPI gene signature;
treating the subject for sepsis by administering to the subject a therapy selected from antibiotics, corticosteroids, immunomodulators, vasopressors and combinations thereof;
obtaining a sample from the subject after the treatment, wherein the sample is blood;
detecting in the sample after the treatment the expression levels of genes in the Drp1-DEGs gene signature and the Drp1-PPI gene signature;
calculating a second sepsis survival score as a linear combination of gene expression values and their corresponding weight values in the Drp1-DEGs gene signature and Drp1-PPI gene signature;
determining a reduced sepsis severity in the subject wherein the second sepsis survival score is higher than the first sepsis survival score, or a maintained or worsened sepsis severity in the subject if the second sepsis survival score is equal to or lower than the first sepsis survival score; and
changing the therapy to a new treatment selected from antibiotics, corticosteroids, immunomodulators, vasopressors and combinations thereof if the second sepsis survival score is equal to, or lower than the first sepsis survival score, or maintaining the therapy if the second sepsis survival score is higher than the first sepsis survival score,
wherein the Drp1-PPI gene signature comprises AFG3L2, BCL2, ITPR3, LONP1, OPTN, PHB, and TOMM40, and
wherein the Drp1-DEGs gene signature comprises SLC4A7, PDCD2L, UPP1, ARL4C, NELL2, LEF1, SGK1, PDE4D, S1PR3, MCTP2, HES6, and OLR1.

2. The method of claim 1, the sample being whole blood, plasma, or serum.

3-5. (canceled)

6. The method of claim 1, each of the first and second sepsis survival scores being calculated using a formula of:
$$sepsis survival score = {\sum }_{i = 1}^{n} {W}_{i} ( \frac{{e}_{i} - {μ}_{i}}{{S}_{i}} ) ,$$
wherein n is the number of genes in the gene signature; Wᵢ is the weighted value of each gene; eᵢ is the expression level of each gene; and μᵢ and Sᵢ are the mean and standard deviation of the gene expression level values, respectively, for the corresponding gene across whole samples, and wherein Wᵢ is 1 for genes AFG3L2, BCL2, ITPR3, LONP1, OPTN, PHB, TOMM40, SLC4A7, PDCD2L, ARL4C, NELL2, LEF1, SGK1, S1PR3, HES6, and OLR1 and is −1 for genes UPP1, PDE4D, and MCTP2.

7. The method of claim 1, further comprising comparing the first and/or the second sepsis survival score with a mean sepsis survival score, and determining the subject as with low sepsis severity wherein the first and/or second sepsis survival score is higher than the mean sepsis survival score.

8. A method of determining efficacy of a therapy for sepsis in a subject in need thereof, wherein the subject is human, comprising the following steps:
(1) obtaining a sample from the subject, detecting in the sample the expression levels of genes in Drp1-DEGs gene signature and Drp1-PPI gene signature, and calculating a first sepsis survival score as a linear combination of gene expression values and their corresponding weight values in the Drp1-DEGs gene signature and the Drp1-PPI gene signature, wherein the sample is blood;
(2) administering the therapy to the subject, wherein the therapy is one or more of antibiotics, corticosteroids, immunomodulators, and vasopressors;
(3) obtaining a sample from the subject after administering the therapy, detecting in the sample the expression levels of genes in the Drp1-DEGs gene signature and the Drp1-PPI gene signature, and calculating a second sepsis survival score as a linear combination of gene expression values and their corresponding weight values in the Drp1-DEGs gene signature and the Drp1-PPI gene signature, wherein the sample is blood; and
(4) determining the therapy as being effective for sepsis in the subject if the second sepsis survival score is higher than the first sepsis survival score, or determining the therapy as being ineffective for sepsis in the subject if the second sepsis survival score is lower than the first sepsis survival score, and changing the therapy to a new treatment selected from antibiotics corticosteroids, immunomodulators, vasopressors and combinations thereof if the second sepsis survival score is lower than the first sepsis survival score, or maintaining the therapy if the second sepsis survival score is higher than the first sepsis survival score,
wherein the Drp1-PPI gene signature comprises AFG3L2, BCL2, ITPR3, LONP1, OPTN, PHB, and TOMM40, and
wherein the Drp1-DEGs gene signature comprises SLC4A7, PDCD2L, UPP1, ARL4C, NELL2, LEF1, SGK1, PDE4D, S1PR3, MCTP2, HES6, and OLR1.

9. The method of claim 8, the samples obtained in step (1) and step (3) being of the same type.

10-12. (canceled)

13. The method of claim 8, each of the first and second sepsis survival scores being calculated using a formula of:
$$sepsis survival score = {\sum }_{i = 1}^{n} {W}_{i} ( \frac{{e}_{i} - {μ}_{i}}{{S}_{i}} ) ,$$
wherein n is the number of genes in the gene signature; Wᵢ is the weighted value of each gene; eᵢ is the expression level of each gene; and μᵢ and Sᵢ are the mean and standard deviation of the gene expression level values, respectively, for the corresponding gene across whole samples, and wherein Wᵢ is 1 for genes AFG3L2, BCL2, ITPR3, LONP1, OPTN, PHB, TOMM40, SLC4A7, PDCD2L, ARL4C, NELL2, LEF1, SGK1, S1PR3, HES6, and OLR1 and is −1 for genes UPP1, PDE4D and MCTP2.

14. (canceled)

15. A method of treating sepsis in a subject in need thereof, wherein the subject is human, comprising the following steps of:
(1) obtaining a sample from the subject, wherein the sample is blood;
(2) detecting in the sample the expression levels of AFG3L2, BCL2, ITPR3, LONP1, OPTN, PHB, and TOMM40, and detecting in the sample the expression levels of SLC4A7, PDCD2L, UPP1, ARL4C, NELL2, LEF1, SGK1, PDE4D, S1PR3, MCTP2, HES6, and OLR1;
(3) calculating a first sepsis survival score as a linear combination of gene expression values and their corresponding weight values;
(4) treating the subject for sepsis by administering to the subject a therapy selected from antibiotics, corticosteroids, immunomodulators, vasopressors and combinations thereof;
(5) obtaining a sample from the subject after the treatment, wherein the sample is blood;
(6) detecting in the sample after the treatment the expression levels of AFG3L2, BCL2, ITPR3, LONP1, OPTN, PHB, and TOMM40, and detecting in the sample the expression levels of SLC4A7, PDCD2L, UPP1, ARL4C, NELL2, LEF1, SGK1, PDE4D, S1PR3, MCTP2, HES6, and OLR1;
(7) calculating a second sepsis survival score as a linear combination of gene expression values and their corresponding weight values;
(8) determining the therapy as a favorable treatment if the second sepsis survival score is higher than the first sepsis survival score, or determining the therapy as an unfavorable treatment if the second sepsis survival score is equal to or lower than the first sepsis survival score; and
(9) changing the therapy to a new ed m a antibiotic corticosteroids, immunomodulators, vasopressors and new combinations thereof if the therapy is an unfavorable treatment, or maintaining the therapy if the therapy is a favorable treatment.

16-18. (canceled)

19. The method of claim 15, each of the first and second sepsis survival scores being calculated using a formula of:
$$sepsis survival score = {\sum }_{i = 1}^{n} {W}_{i} ( \frac{{e}_{i} - {μ}_{i}}{{S}_{i}} ) ,$$
wherein n is the number of genes in the gene signature; Wᵢ is the weighted value of each gene; eᵢ is the expression level of each gene; and μᵢ and Sᵢ are the mean and standard deviation of the gene expression level values, respectively, for the corresponding gene across whole samples, and wherein Wᵢ is 1 for genes AFG3L2, BCL2, ITPR3, LONP1, OPTN, PHB, TOMM40, SLC4A7, PDCD2L, ARL4C, NELL2, LEF1, SGK1, S1PR3, HES6, and OLR1 and is −1 for genes UPP1, PDE4D, and MCTP2.

20. The method of claim 15, the samples obtained in steps (1) and (5) being of the same type.

21-23. (canceled)
